SEQUENCE LISTING (1 sequence)

SEQ ID NO: 1 - protein, 11 aa, Artificial
  Human protein analogue
CQIWKQKPDLC

(19) United States
(12) Patent Application Publication
(10) Pub. No.: US 2008/0227724 A1
(43) Pub. Date: Sep. 18, 2008

(54) Anti-Inflammatory Agents

(75) Inventors: David John Grainger, Cambridge (GB); David John Fox, Cambridge (GB)

Correspondence Address: JACOBSON HOLMAN PLLC, 400 SEVENTH STREET N.W., SUITE 600, WASHINGTON, DC, 20004, US

(73) Assignee: CAMBRIDGE UNIVERSITY TECHNICAL SERVICES LIMITED, Cambridge (GB)

(21) Appl. No.: 10/581,274

(22) PCT Filed: Nov. 30, 2004
(86) PCT No.: PCT/GB2004/005030

§ 371 Date: May 30, 2008

(30) Foreign Application Priority Data
Dec. 1, 2003 (GB) 0327775.3
Aug. 5, 2004 (GB) 0417436.3
Aug. 10, 2004 (GB) 0417734.1

(51) Int. Cl. C07D 223/10; C07K 5/06; C07K 5/08; A61K 38/05; C07C 69/007; C07C 53/00; A61P 37/00; A61P 25/00; A61P 35/00; A61P 19/02; A61P 31/12; C07D 263/02; C07C 53/15; A61K 38/06; A61K 38/07; A61K 31/55

(52) U.S. Cl. 514/18; 540/531; 514/212.03; 514/19; 530/331; 530/330; 560/129; 562/602; 562/512; 548/215; 549/356

(57) ABSTRACT

The invention relates to the use of 3-aminocaprolactam derivatives for preparing a medicament intended to prevent or treat inflammatory disorders, and uses compounds of general formula (I) or a pharmaceutically acceptable salts thereof;

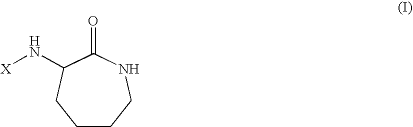

wherein
X is —CO—R¹ or —SO₂—R², and R¹ and R² are carbonaceous substituents.

The invention relates to the use of 3-aminocaprolactam derivatives for preparing a medicament intended to prevent or treat inflammatory disorders.

Inflammation is an important component of physiological host defense. Increasingly, however, it is clear that temporally or spatially inappropriate inflammatory responses play a part in a wide range of diseases, including those with an obvious leukocyte component (such as autoimmune diseases, asthma or atherosclerosis) but also in diseases that have not traditionally been considered to involve leukocytes (such as osteoporosis or Alzheimer's disease).

The chemokines are a large family of signalling molecules with homology to interleukin-8 which have been implicated in regulating leukocyte trafficking both in physiological and pathological conditions. With more than fifty ligands and twenty receptors involved in chemokine signalling, the system has the requisite information density to address leukocytes through the complex immune regulatory processes from the bone marrow, to the periphery, then back through secondary lymphoid organs. However, this complexity of the chemokine system has at first hindered pharmacological approaches to modulating inflammatory responses through chemokine receptor blockade. It has proved difficult to determine which chemokine receptor(s) should be inhibited to produce therapeutic benefit in a given inflammatory disease.

More recently, a family of agents which block signalling by a wide range of chemokines simultaneously has been described: Reckless et al., Biochem J. (1999) 340:803-811. The first such agent, a peptide termed “Peptide 3”, was found to inhibit leukocyte migration induced by 5 different chemokines, while leaving migration in response to other chemoattractants (such as fMLP or TGF-beta) unaltered. This peptide, and its analogs such as NR58-3.14.3 (i.e. Sequence ID No. 1 c(DCys-DGln-DIle-DTrp-DLys-DGln-DLys-DPro-DAsp-DLeu-DCys)-NH₂), are collectively termed “Broad Spectrum Chemokine Inhibitors” (BSCIs). Grainger et al., Biochem. Pharm. 65 (2003) 1027-1034 have subsequently shown BSCIs to have potentially useful anti-inflammatory activity in a range of animal models of diseases. Interestingly, simultaneous blockade of multiple chemokines is not apparently associated with acute or chronic toxicity, suggesting this approach may be a useful strategy for developing new anti-inflammatory medications with similar benefits to steroids but with reduced side-effects.

However, peptides and peptoid derivatives such as NR58-3.14.3, may not be optimal for use in vivo. They are quite expensive to synthesise and have relatively unfavourable pharmacokinetic and pharmacodynamic properties. For example, NR58-3.14.3 is not orally bioavailable and is cleared from blood plasma with a half-life period of less than 30 minutes after intravenous injection.

Two parallel strategies have been adopted to identify novel preparations which retain the anti-inflammatory properties of peptide 3 and NR58-3.14.3, but have improved characteristics for use as pharmaceuticals. Firstly, a series of peptide analogs have been developed, some of which have longer plasma half-lives than NR58-3.14.3 and which are considerably cheaper to synthesise. Secondly, a detailed structure: activity analysis of the peptides has been carried out to identify the key pharmacophores and design small non-peptidic structures which retain the beneficial properties of the original peptide.

This second approach yielded several structurally distinct series of compounds which retained the anti-inflammatory properties of the peptides, including 16-amino and 16-aminoalkyl derivatives of the alkaloid yohimbine, as well as a range of N-substituted 3-aminoglutarimides. (Reference: Fox et al., J Med Chem 45 (2002) 360-370: WO 99/12968 and WO 00/42071.) All of these compounds are broad-spectrum chemokine inhibitors which retain selectivity over non-chemokine chemoattractants, and a number of them have been shown to block acute inflammation in vivo.

The most potent and selective of these compounds was (S)-3-(undec-10-enoyl)-aminoglutarimide (NR58,4), which inhibited chemokine-induced migration in vitro with an ED₅₀ of 5 nM. However, further studies revealed that the aminoglutarimide ring was susceptible to enzymatic ring opening in serum. Consequently, for some applications (for example, where the inflammation under treatment is chronic, such as in autoimmune diseases) these compounds may not have optimal properties, and a more stable compound with similar anti-inflammatory properties may be superior.

As an approach to identifying such stable analogs, various derivatives of (S)-3-(undec-10-enoyl)-aminoglutarimide have been tested for their stability in serum. One such derivative, the 6-deoxo analog (S)-3-(undec-10-enoyl)-tetrahydropyridin-2-one, is completely stable in human serum for at least 7 days at 37° C., but has considerably reduced potency compared with the parental molecule.

Amide derivatives of 3-aminocaprolactam have already been disclosed in the art. For example:
Japanese patent application No. 09087331 describes 3-aminocaprolactam amide derivatives wherein the amide alkyl side chain may contain from 2 to 30 carbon atoms. These compounds have been presented as oil-gelating agents.
U.S. Pat. No. 6,395,282 describes immunogenic conjugates comprising a carrier molecule coupled to an autoinducer of a Gram negative bacteria, wherein said autoinducer can be a 3-aminocaprolactam amide derivative wherein the amide alkyl side chain may contain up to 34 carbon atoms. However, a therapeutic use is disclosed only for the conjugates and not for the isolated amide derivative.
An article by Weiss et al. (Research Communications in Psychology, Psychiatry and Behavior (1992), 17(3-4), 153-159) discloses a series of 3-aminocaprolactam amide derivatives, and among others 3-hexanamido-DL-ε-caprolactam and 3-dodecanamido-DL-ε-caprolactam. These compounds are presented as having only an in vitro activity but no significant in vivo effect.

In other words, though some alkyl amide derivatives of 3-aminocaprolactam have certainly been known in the art, no actual pharmaceutical use has been described for 3-aminocaprolactam amide derivatives.

The invention provides the use of a compound of general formula (I), or a pharmaceutically acceptable salt thereof, for the preparation of a medicament intended to treat inflammatory disorder:

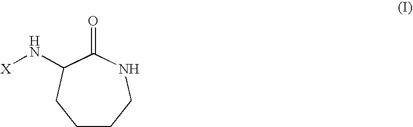

wherein

### X is —CO—R¹ or —SO₂—R²,

R¹ is an alkyl, haloalkyl, alkoxy, haloalkoxy, alkenyl, alkynyl or alkylamino radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms); and
R² is an alkyl radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, and of 13 to 17 carbon atoms).

Alternatively R¹ and R² may be selected independently from a peptido radical, for example having from 1 to 4 peptidic moieties linked together by peptide bonds (for example a peptido radical of 1 to 4 amino acid residues).

The carbon atom at position 3 of the caprolactam ring is asymmetric and consequently, the compounds according to the present invention have two possible enantiomeric forms, that is, the “R” and “S” configurations. The present invention encompasses the two enantiomeric forms and all combinations of these forms, including the racemic “RS” mixtures. With a view to simplicity, when no specific configuration is shown in the structural formulae, it should be understood that the two enantiomeric forms and their mixtures are represented.

GB priority applications 0327775.3 and 0417436.3 by the same applicant on 3-amino-caprolactam compounds have indicated correctly that ‘S’-configuration compounds are preferred—these applications wrongly illustrate a general Formula (I′) showing the ‘R’-configuration.

Preferably, the compounds of general formula (I) or pharmaceutically acceptable salts thereof used according to this aspect of the invention will be compounds of general formula (I′)

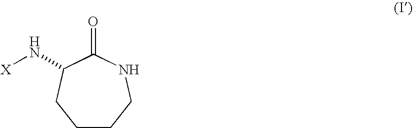

wherein X has the same meaning as above.

The carbon atoms in R¹ and R² may be linear or branched.

The compounds of general formula (I) or (I′), or their pharmaceutically acceptable salts, may be such that the alkyl, haloalkyl, alkoxy, haloalkoxy, alkenyl, alkynyl or alkylamino part of the R¹ radical is either linear or is branched but contains a linear chain of at least 8 or at least 10 carbon atoms.

The invention also provides pharmaceutical compositions comprising, as active ingredient, a compound of general formula (I), or a pharmaceutically acceptable salt thereof, and at least one pharmaceutically acceptable excipient and/or carrier:

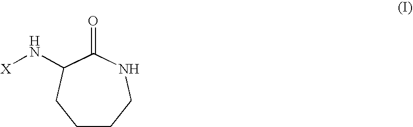

wherein

### X is —CO—R¹ or —SO₂—R²,

R¹ is an alkyl, haloalkyl, alkoxy, haloalkoxy, alkenyl, alkynyl or alkylamino radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms); and
R² is an alkyl radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, and of 13 to 17 carbon atoms).

Alternatively R¹ and R² may be selected independently from a peptido radical, for example having from 1 to 4 peptidic moieties linked together by peptide bonds (for example a peptido radical of 1 to 4 amino acid residues).

Preferably, the compounds of general formula (I) or pharmaceutically acceptable salts thereof used according to this aspect of the invention will be compounds of general formula (I′)

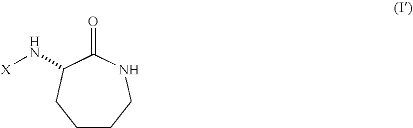

wherein X has the same meaning as above.

By pharmaceutically acceptable salt is meant in particular the addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Also within the scope of the present invention, when they can be used, are the salts formed from bases such as sodium or potassium hydroxide. For other examples of pharmaceutically acceptable salts, reference can be made to “Salt selection for basic drugs”, Int. J. Pharm. (1986), 33, 201-217.

The pharmaceutical composition can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax. Other appropriate pharmaceutically acceptable excipients and/or carriers will be known to those skilled in the art.

The pharmaceutical compositions according to the invention can also be presented in liquid form, for example, solutions, emulsions, suspensions or syrups. Appropriate liquid supports can be, for example, water, organic solvents such as glycerol or glycols, as well as their mixtures, in varying proportions, in water.

The invention also provides compounds and salts thereof of general formula (I)

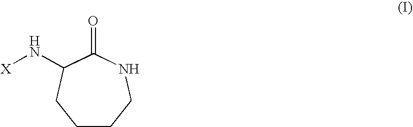

wherein

### X is —CO—R¹ or —SO₂—R²,

R¹ is an alkyl, haloalkyl, alkoxy, haloalkoxy, alkenyl, alkynyl or alkylamino radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms); and
R² is an alkyl radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, and of 13 to 17 carbon atoms).

Alternatively R¹ and R² may be selected independently from a peptido radical, for example having from 1 to 4 peptidic moieties linked together by peptide bonds (for example a peptido radical of 1 to 4 amino acid residues).

Preferably, the compounds of general formula (I) or salts thereof used according to this aspect of the invention will be compounds of general formula (I′)

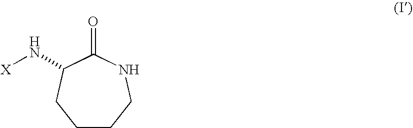

wherein X has the same meaning as above.

Preferably, the compounds of general formula (I) or (I′) when used in the invention, or their salts, will be such that the alkyl, haloalkyl, alkoxy, haloalkoxy, alkenyl, alkynyl or alkylamino part of the R¹ radical is either linear or is branched but contains a linear chain of at least 8 or 10 carbon atoms.

In particular, preferred compounds of general formula (I) or (I′) and their salts according to any aspect of the present invention are selected from the group consisting of:
(S)-3-hexadecanoylamino-caprolactam;
(S)-3-undecanoylamino-caprolactam;
(S)-3-(undec-10-enoyl)amino-caprolactam;
(S)-3-(undec-10-ynoyl)amino-caprolactam;
(S)-3-tetradecanoylamino-caprolactam;
(R)-3-hexadecanoylamino-caprolactam;
(S)-3-octadecanoylamino-caprolactam;
(S)-(Z)-3-(hexadec-9-enoyl)amino-caprolactam;
(S)-(Z)-3-(octadec-9-enoyl)amino-caprolactam;
(R)-(Z)-3-(octadec-9-enoyl)amino-caprolactam;
(S)-3-(2′,2′-dimethyl-dodecanoyl)amino-caprolactam;
(S)-3-(decyloxycarbonyl)amino-caprolactam;
(S)-(E)-3-(dodec-2-enoyl)amino-caprolactam;
(S)-3-(dec-9-enylaminocarbonyl)amino-caprolactam;
(S)-3-(decylaminocarbonyl)amino-caprolactam;
   and the salts thereof.

The most preferred compounds will be selected from the group consisting of: (S)-3-hexadecanoylamino-caprolactam (i.e. the compound of general formula (I′) wherein R¹ is hexadecanyl), (S)-3-(2′,2′-dimethyl-dodecanoyl)amino-caprolactam (S)-3-(2′,2′-dimethyl-propionyl)amino-caprolactam and salts thereof.

As mentioned in the discussion of prior art above, certain alkyl amide derivatives of 3-amino caprolactam may be known as compounds per se (though it is not presently known that any have been described as such as pharmaceutical compositions or for medical use in an anti-inflammatory context). There may be in the prior art disclosure of straight chain alkyl amide derivatives of 3-amino caprolactam. In so far as any compound in known as such, this compound is not intended to be a compound claimed per se in this invention, and is hereby disclaimed. Consequently the applicant explicitly distinguishes herein between straight chain alkyl derivatives covered by the definition of formula (I) and (I′) herein, and branched chain alkyl derivatives of formula (I) and (I′) herein. The definition of R¹ used herein in connection with compounds per se may included all alkyl derivatives; alternatively R¹ may include all alkyl derivatives with the exception of certain specified straight chain alkyl derivatives; alternatively R¹ may include all branched chain alkyl derivatives; and as a further alternative the definition of R¹ may exclude all alkyl amide derivatives of 3-amino caprolactam.

The invention includes compounds, compositions and uses thereof as defined, wherein the compound is in hydrated or solvated form.

As indicated in the Introduction, certain alkyl aminocaprolactam compounds per se, and compositions/conjugates containing them, may already be known in the prior art. Any such known compounds or compositions will be disclaimed from the present invention, either by specific disclaimer or by generic disclaimer of a class of compounds/compositions.

The amide derivatives of 3-aminocaprolactam described here are functional BSCIs. They are relatively inexpensive to synthesise, using facile synthesis routes provided herein; they are stable in human serum and consequently have excellent pharmacokinetic properties; they are orally bioavailable; they are highly potent broad-spectrum chemokine inhibitors in vitro with excellent selectivity over non-chemokine chemoattractants; they are highly potent and effective anti-inflammatory agents in vivo in rodent models of inflammation; their administration is not associated with any significant acute toxicity at the doses necessary to achieve a maximal therapeutic effect. Taken together, these properties suggest that amide derivatives of 3-aminocaprolactam represent, anti-inflammatory medications with advantages over previously described compounds.

In comparison to the prior art the improvement of the present invention lies in the introduction of the aminocaprolactam moiety. However, the chemical structure of the side chain (whether alkyl amide, alkyl sulfonamide or peptido) can also significantly affect the properties of the molecule, such that alkyl substituents with substitution at the 2-position (relative to the amide carbonyl) or 1-position (relative to the sulfonamide sulfonyl group) are significantly superior to compounds with linear alkyl chains (whether alkyl amides or alkyl sulfonamides).

Prior art peptides (such as NR58-3.14.3) have the disadvantages that: (a) they are expensive and require solid phase synthesis (at least for the longer ones) and (b) they clear very quickly via the kidneys and (c) they are generally less potent.

The prior art aminoglutarimides are cheap, not cleared quickly via the kidneys and more potent BUT they do not show metabolic stability.

The improvement described here, the aminocaprolactams, are cheap, not cleared by the kidney and even more potent, and are also metabolically stable.

According to this invention, inflammatory disorders intended to be prevented or treated by the compounds of general formula (I) or (I′) or the pharmaceutically acceptable salts thereof or pharmaceutical compositions or medicaments containing them as active ingredients include notably:
autoimmune diseases, for example such as multiple sclerosis;
vascular disorders including stroke, coronary artery diseases, myocardial infarction, unstable angina pectoris, atherosclerosis or vasculitis, e.g., Behçet's syndrome, giant cell arteritis, polymyalgia rheumatica, Wegener's granulomatosis, Churg-Strauss syndrome vasculitis, Henoch-Schönlein purpura and Kawasaki disease;
viral infection or replication, e.g. infections due to or replication of viruses including pox virus, herpes virus (e.g., Herpesvirus samiri), cytomegalovirus (CMV) or lentivirus;
asthma;
osteoporosis; (low bone mineral density);
tumor growth;
rheumatoid arthritis;
organ transplant rejection and/or delayed graft or organ function, e.g. in renal transplant patients;
a disorder characterised by an elevated TNF-α level;
psoriasis;
skin wounds;
disorders caused by intracellular parasites such as malaria or tuberculosis;
allergies; or
Alzheimer's disease.

According to this invention, further inflammatory disorders include:
ALS;
fibrosis (particularly pulmonary fibrosis, but not limited to fibrosis in the lung);
the formation of adhesions (particularly in the peritoneum and pelvic region).
antigen induced recall response
immune response suppression

These clinical indications fall under the general definition of inflammatory disorders or disorders characterized by elevated TNFα levels.

Where legally permissible, the invention also provides a method of treatment, amelioration or prophylaxis of the symptoms of an inflammatory disease (including an adverse inflammatory reaction to any agent) by the administration to a patient of an anti-inflammatory amount of a compound, composition or medicament as claimed herein.

Administration of a medicament according to the invention can be carried out by topical, oral, parenteral route, by intramuscular injection, etc.

The administration dose envisaged for a medicament according to the invention is comprised between 0.1 mg and 10 g depending on the type of active compound used.

According to the invention, the compounds of general formula (I) or (I′) can be prepared using the processes described hereafter.

### Preparation of the Compounds of General Formula (I) or (I′)

All the compounds of general formula (I′) or (I′) can be prepared easily according to general methods known to the person skilled in the art.

Nevertheless, the following preferred synthetic routes are proposed:

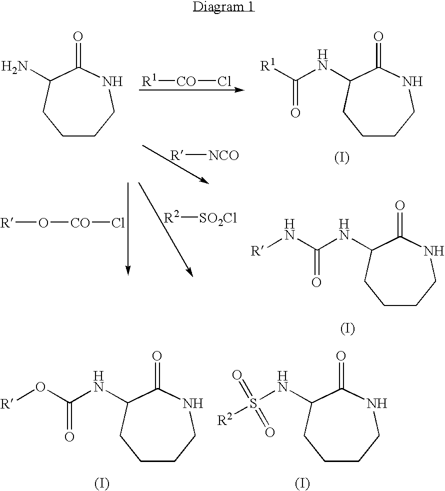

According to the routes shown in Diagram 1:
3-amino-caprolactam is treated by an acid chloride of general formula R¹—CO—Cl wherein R¹ is an alkyl, haloalkyl, alkenyl or alkynyl radical to produce the compounds of general formula (I) wherein X is —CO—R¹ and R¹ is an alkyl, haloalkyl, alkenyl or alkynyl radical; or
3-amino-caprolactam is treated by an isocyanate of general formula R′—NCO wherein R′ is alkyl to produce the compounds of general formula (I) wherein X is —CO—R¹ and R¹ is an alkylamino radical;
3-amino-caprolactam is treated by a sulphochloride of general formula R²—SO₂Cl wherein R² is alkyl to produce the compounds of general formula (I) wherein X is —SO₂—R² and R² is an alkyl radical; or
3-amino-caprolactam is treated by a chloroformate of general formula R′—O—CO—Cl wherein R′ is alkyl to produce the compounds of general formula (I) wherein X is —CO—R¹ and R¹ is an alkoxy radical.

The reactions shown in Diagram 1 may be carried out, for example, in chloroform or dichloromethane. The most preferred reaction solvent is dichloromethane.

The above reactions are preferably carried out in the presence of a base, for example Na₂CO₃.

All the above reactions may be carried out at ambient temperature (about 25° C.) or more generally at a temperature between 20 and 50° C.

### DEFINITIONS

The term “about” refers to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range. Hence, for example, the range of 4 to 20 carbon atoms specified in respect of (inter alia) formula I is intended to include all integers between 4 and 20 and all sub-ranges of each combination of upper and lower numbers, whether exemplified explicitly or not.

As used herein, the term “comprising” is to be read as meaning both comprising and consisting of Consequently, where the invention relates to a “pharmaceutical composition comprising as active ingredient” a compound, this terminology is intended to cover both compositions in which other active ingredients may be present and also compositions which consist only of one active ingredient as defined.

The term “peptidic moieties” used herein is intended to include the following 20 naturally-occurring proteogenic amino acid residues:

| SYMBOL | MEANING |
|---|---|
| Ala | Alanine |
| Cys | Cysteine |
| Asp | Aspartic Acid |
| Glu | Glutamic Acid |
| Phe | Phenylalanine |
| Gly | Glycine |
| His | Histidine |
| Ile | Isoleucine |
| Lys | Lysine |
| Leu | Leucine |
| Met | Methionine |
| Asn | Asparagine |
| Pro | Proline |
| Gln | Glutamine |
| Arg | Arginine |
| Ser | Serine |
| Thr | Threonine |
| Val | Valine |
| Trp | Tryptophan |
| Tyr | Tyrosine |

Modified and unusual amino acid residues, as well as peptido-mimetics, are also intended to be encompassed within the definition of “peptidic moieties”.

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

The following examples are presented in order to illustrate the above procedures and should in no way be considered to limit the scope of the invention.

### FIGURES

FIG. 1 provides a comparison of (R)- and (S)-enantiomers of amide derivatives of aminocaprolactam as inhibitors of MCP-1 induced migration.

### EXAMPLES

### General Procedure for the Synthesis of the Starting Compounds

The hydrochlorides of (R) and (S)-3-amino-caprolactam, and the hydro-pyrrolidine-5-carboxylates of (R,R) and (S,S)-3-amino-caprolactam were synthesised according to literature (cf. Boyle et al., J. Org. Chem., (1979), 44, 4841-4847; Rezler et al., J. Med. Chem. (1997), 40, 3508-3515).

### Example 1

### (S)-3-hexadecanoylamino-caprolactam

(S)-3-amino-caprolactam hydrochloride (5 mmol) and Na₂CO₃ (15 mmol) in water (25 ml) were added to a solution of hexadecanoyl chloride (5 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over N % CO₃ and reduced in vacuo. The residue was purified by recrystallisation from EtOAc to give the title compound (1.41 g; 77%).

Melting point: 99-100° C.

[α]_{D}²⁵ (c=1, CHCl₃)=+32.0.

IR: νₘₐₓ (cm⁻¹): 3325, 3272 (NH), 1666, 1655, 1631 (CO), 1524 (NH).

¹H NMR (δ_{H}, 500 MHz, CDCl₃): 6.88 (1H, d, J 5.5, CHNH), 6.72 (1H, br s, CH₂NH), 4.49 (1H, ddd, J 11, 6, 1, CHNH), 3.29-3.16 (2H, m, CH₂NH), 2.17 (2H, t, J 7.5, CH₂CONH), 2.03 (1H, br d, J 13.5, ring CH), 1.98-1.89 (1H, m, ring CH), 1.85-1.73 (2H, m, ring CH), 1.58 (2H, br qn J 7.0, CH₂CH₂CONH), 1.43 (1H, br qd, J 14, 3, ring CH), 1.38-1.29 (1H, br m, ring CH), 1.29-1.14 (24H, m, (CH₂)₁₂) and 0.83 (3H, t, J 6.5, CH₃).

¹³C NMR (δ_{C}, 125 MHz, CDCl₃): 175.9, 172.3 (CO), 52.0 (NHCHCO), 42.1 (NCH₂), 36.6, 31.9, 31.7, 29.6 (×6), 29.4, 29.3 (×2), 29.2, 28.8, 27.9, 25.6, 22.6 (CH₂) and 14.1 (CH₃).

m/z (C₂₂H₄₂N₂O₂Na): 389.31450 (calculated: 389.3144).

### Example 2

### (S)-3-undecanoylamino-caprolactam

(S)-3-amino-caprolactam hydrochloride (2 mmol) and Na₂CO₃ (6 mmol) in water (25 ml) were added to a solution of undecanoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (397 mg, 67%).

Melting point: 91-92° C.

[α]_{D}²⁵ (c=1, CHCl₃)=+30.2.

IR: νₘₐₓ (cm⁻¹): 3342, 3313 (NH), 1676, 1638 (CO), 1519 (NH); 3342, 3292 (NH), 1671, 1639 (CO), 1513 (NH).

¹H NMR (δ_{H}, 500 MHz, d₆-DMSO): 7.76 (1H, t, J 6, CH₂NH), 7.68 (1H, d, J 7, CHNH), 4.38 (1H, dd, J 10, 7, CHNH), 3.15 (1H, ddd, J 15.5, 11, 5, CHHNH), 3.04 (1H, dt, J 13, 6, CHHNH), 2.19-2.06 (2H, m, CH₂CONH), 1.85 (1H, dt, J 10.5, 3, C-5H), 1.77-1.68 (2H, m, C-4H, C-6H), 1.60 (1H, qt, J 12, 3.5, C-5H), 1.46 (2H, br qn J 6.5, CH₂CH₂CONH), 1.35 (1H, qd, J 12.5, 3, C-4H), 1.31-1.13 (15H, m, (CH₂)₇+C-6H) and 0.85 (3H, t, J 7.0, CH₃).

¹³C NMR (δ_{C}, 125 MHz, d₆-DMSO): 174.4 (CO-ring), 171.3 (CO-chain), 51.3 (NHCHCO), 40.7 (NCH₂), 35.2, 31.4, 31.3, 29.1, 29.0 (×2), 28.9, 28.8, 28.7, 27.8, 25.4, 22.2 (CH₂) and 14.0 (CH₃).

m/z (C₁₇H₃₂N₂O₂Na): 319.23540 (calculated: 319.2361).

### Example 3

### (S)-3-(undec-10-enoyl)amino-caprolactam

(S)-3-amino-caprolactam hydrochloride (2 mmol) and Na₂CO₃ (6 mmol) in water (25 ml) were added to a solution of undec-10-enoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (423 mg; 72%).

Melting point: 83-84° C.

[α]_{D}²⁵ (c=1, CHCl₃)=+40.1.

IR: νₘₐₓ (cm⁻¹): 3327, 3273 (NH), 1655, 1630 (CO), 1521 (NH).

¹H NMR (δ_{H}, 500 MHz, d₆-DMSO): 7.75 (1H, t, J 6, CH₂NH), 7.66 (1H, d, J 7, CHNH), 5.76 (1H, ddt, J 17, 10, 6.5 CH₂═CH), 4.96 (1H, dq, J 17, 2, CHH═CH), 4.96 (1H, ddt, J 17, 2, 1, CHH═CH), 4.36 (1H, dd, J 10, 7, CHNH), 3.14 (1H, ddd, J 15.5, 11.5, 5, CHHNH), 3.03 (1H, br dt, J 13, 5.5, CHHNH), 2.16-2.06 (2H, m, CH₂CONH), 1.98 (2H, br q, J 7, CH₂═CHCH₂), 1.85 (1H, dt, J 10.5, 3, C-5H), 1.75-1.67 (2H, m, C-4H, C-6H), 1.60 (1H, qt, J 13, 3.5, C-5H), 1.44 (2H, br qn, J 7, CH₂CH₂CONH), 1.39-1.27 (3H, m, CH₂═CHCH₂CH₂+C-4H) and 1.31-1.13 (9H, m, (CH₂)₄+C-6H).

¹³C NMR (δ_{C}, 125 MHz, d₆-DMSO): 174.4 (CO-ring), 171.3 (CO-chain), 138.9 (CH₂═CH), 114.7 (CH₂═CH), 51.3 (NHCHCO), 40.7 (NCH₂), 35.3, 33.3, 31.3, 29.0, 28.9 (×2) 28.7, 28.6, 28.4, 27.8 and 25.4 (CH₂). m/z (C₁₇H₃₀N₂O₂Na): 317.21970 (calculated: 317.2205).

### Example 4

### (S)-3-(undec-10-ynoyl)amino-caprolactam

(S)-3-amino-caprolactam hydrochloride (2 mmol) and Na₂CO₃ (6 mmol) in water (25 ml) were added to a solution of undec-10-ynoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (362 mg; 62%).

Melting point: 73-75° C.

[α]_{D}²⁵ (c=1, CHCl₃)=+42.1.

IR: νₘₐₓ (cm⁻¹): 3332, 3295 (NH), 1667, 1633 (CO), 1523 (NH).

¹H NMR (δ_{H}, 500 MHz, d₆-DMSO): 7.76 (1H, t, J 5.5, CH₂NH), 7.68 (1H, d, J 7, CHNH), 4.36 (1H, dd, J 11, 7, CHNH), 3.16 (1H, ddd, J 15.5, 11.5, 5, CHHNH), 3.03 (1H, br dt, J 14, 7, CHHNH), 2.17-2.07 (4H, m, CH₂CONH+CH₂CCH), 1.85 (1H, m, C-5H), 1.77-1.67 (2H, m, C-4H, C-6H), 1.62 (1H, br qt, J 13, 3.0, C-5H), 1.50-1.28 (5H, m, CH₂CH₂CONH+HCCCH₂CH₂+C-4H) and 1.28-1.13 (9H, m, (CH₂)₄+C-6H).

¹³C NMR (δ_{C}, 125 MHz, d₆-DMSO): 174.4 (CO-ring), 171.3 (CO-chain), 84.6 (CH₂CCH), 71.1 (CH₂CCH), 51.3 (NHCHCO), 40.7 (NCH₂), 35.2, 31.3, 29.0, 28.8, 28.7, 28.5, 28.2, 28.0, 27.8, 25.4 and 17.8 (CH₂).

m/z (C₁₇H₂₈N₂O₂Na): 317.20470 (calculated: 315.2048).

### Example 5

### (S)-3-dodecanoylamino-caprolactam

(S)-3-amino-caprolactam hydrochloride (2 mmol) and Na₂CO₃ (6 mmol) in water (25 ml) were added to a solution of dodecanoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (439 mg, 71%).

Melting point: 93-94° C.

[α]_{D}²⁵ (c=1, CHCl₃)=+35.5.

IR: νₘₐₓ (cm⁻¹): 3324, 3267 (NH), 1666, 1630 (CO), 1521 (NH).

¹H NMR (δ_{H}, 500 MHz, d₆-DMSO): 7.76 (1H, br s, CH₂NH), 7.67 (1H, d, J 7, CHNH), 4.38 (1H, dd, J 10.5, 7.5, CHNH), 3.15 (1H, ddd, J 15.5, 11.5, 5, CHNH), 3.05 (1H, dt, J 14.5, 5.5, CHHNH), 2.17-2.07 (2H, m, CH₂CONH), 1.90-1.80 (1H, m, C-5H), 1.77-1.68 (2H, m, C-4H, C-6H), 1.62 (1H, br qt. J 12, 3.5, C-5H), 1.46 (2H, br qn J 6.0, CH₂CH₂CONH), 1.36 (1H, qd, J 12.5, 2.5, C-4H), 1.31-1.13 (17H, m, (CH₂)₈+C-6H) and 0.85 (3H, t, J 6.5, CH₃).

¹³C NMR (δ_{C}, 125 MHz, d₆-DMSO): 174.4 (CO-ring), 171.2 (CO-chain), 51.3 (NHCHCO), 40.7 (NCH₂), 35.3, 31.4, 31.3, 29.1 (×3), 29.0 (×2), 28.8, 28.7, 27.8, 25.4, 22.2 (CH₂) and 14.0 (CH₃).

m/z (C₁₈H₃₄N₂O₂Na): 333.25150 (calculated: 333.2518).

### Example 6

### (S)-3-tetradecanoylamino-caprolactam

(S)-3-amino-caprolactam hydrochloride (2 mmol) and Na₂CO₃ (6 mmol) in water (25 ml) were added to a solution of tetradecanoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (412 mg; 61%).

Melting point: 97-98° C.

[α]_{D}²⁵ (c=1, CHCl₃)=+33.2.

IR: νₘₐₓ (cm⁻¹): 3326, 3273 (NH), 1666, 1655, 1631 (CO), 1523 (NH).

¹H NMR (δ_{H}, 500 MHz, CDCl₃): 6.87 (1H, d, J 5.5, CHNH), 6.66-6.48 (1H, br m, CH₂NH), 4.50 (1H, dd, J 11, 6, CHNH), 3.30-3.16 (2H, m, CH₂NH), 2.18 (2H, t, J 7.5, CH₂CONH), 2.04 (1H, br d, J 13.5, ring CH), 2.00-1.92 (1H, m, ring CH), 1.86-1.74 (2H, m, ring CH), 1.59 (2H, br qn J 7.0, CH₂CH₂CONH), 1.43 (1H, br q, J 12.5, ring CH), 1.31 (1H, br q, J 13, ring CH), 1.31-1.13 (20H, m, (CH₂)₁₀) and 0.85 (3H, t, J 6.5, CH₃).

¹³C NMR (δ_{C}, 125 MHz, CDCl₃): 175.9, 172.3 (CO), 52.0 (NHCHCO), 42.1 (NCH₂), 36.6, 31.9, 31.7, 29.6 (×4), 29.4, 29.3 (×2), 29.2, 28.8, 27.9, 25.6, 22.6 (CH) and 14.1 (CH₃).

m/z (C₂₀H₃₈N₂O₂Na): 361.28270 (calculated: 361.2831).

### Example 7

### (R)-3-hexadecanoylamino-caprolactam

(R,R)-3-amino-caprolactam hydro-pyrrolidine-5-carboxylate (5 mmol) and Na₂CO₃ (15 mmol) in water (25 ml) were added to a solution of hexadecanoyl chloride (5 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (1.23 g; 67%).

Melting point: 99-100° C.

[α]_{D}²⁵ (c=1, CHCl₃)=−32.0.

### Example 8

### (S)-3-octadecanoylamino-caprolactam

(S)-3-amino-caprolactam hydrochloride (2 mmol) and Na₂CO₃ (6 mmol) in water (25 ml) were added to a solution of octadecanoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give the title compound (648 mg; 82%).

Melting point: 87-88° C.

[α]_{D}²⁵ (c=1, CHCl₃)=+31.9.

IR: νₘₐₓ (cm⁻¹): 3327, 3272 (NH), 1667, 1655, 1631 (CO), 1524 (NH).

¹H NMR (δ_{H}, 500 MHz, CDCl₃): 6.88 (1H, d, J 5.5, CHNH), 6.72-6.58 (1H, br m, CH₂NH), 4.50 (1H, dd, J 11, 6, CHNH), 3.29-3.16 (2H, m, CH₂NH), 2.17 (2H, t, J 7.5, CH₂CONH), 2.03 (1H, br d, J 13, ring CH), 1.99-1.90 (1H, m, ring CH), 1.86-1.73 (2H, m, ring CH), 1.58 (2H, br qn J 7.0, CH₂CH₂CONH), 1.42 (1H, br qd, J 14, 3, ring CH), 1.38-1.30 (1H, br m, ring CH), 1.30-1.14 (28H, m, (CH₂)₁₄) and 0.84 (3H, t, J 6.5, CH₃).

¹³C NMR (δ_{C}, 125 MHz, CDCl₃): 175.9, 172.3 (CO), 52.0 (NHCHCO), 42.1 (NCH₂), 36.6, 31.9, 31.7, 29.6 (×8), 29.4, 29.3 (×2), 29.2, 28.8, 27.9, 25.6, 22.6 (CH₂) and 14.1 (CH₃).

m/z (C₂₄H₄₆N₂O₂Na): 417.34460 (calculated: 417.3457).

### Example 9

### (S)-(Z)-3-(hexadec-9-enoyl)amino-caprolactam

(S,S)-3-amino-caprolactam hydro-pyrrolidine-5-carboxylate (2 mmol) and Na₂CO₃ (6 mmol) in water (25 ml) were added to a solution of (Z)-hexadec-9-enoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (406 mg; 56%).

Melting point: 67-68° C.

[α]_{D}²⁵ (c=1, CHCl₃)=+33.2.

IR: νₘₐₓ (cm⁻¹): 3324, 3268 (NH), 1655, 1630 (CO), 1524 (NH).

¹H NMR (δ_{H}, 500 MHz, CDCl₃): 6.88 (1H, d, J 5.5, CHNH), 6.67 (1H, br s, CH₂NH), 5.33-5.25 (2H, m, CH═CH), 4.50 (1H, ddd, J 11, 6, 1, CHNH), 3.29-3.16 (2H, m, CH₂NH), 2.17 (2H, t, J 7.5, CH₂CONH), 2.03 (1H, br d, J 13, ring CH), 1.99-1.90 (5H, m, ring CH+CH₂CH═CHCH₂), 1.84-1.72 (2H, m, ring CH), 1.58 (2H, br qn J 7.0, CH₂CH₂CONH), 1.43 (1H, br qd, J 14, 3, ring CH), 1.38-1.30 (1H, br m, ring CH), 1.30-1.14 (16H, m, (CH₂)₄CH₂CH═CHCH₂(CH₂)₄) and 0.84 (3H, t, J 7, CH₃).

¹³C NMR (δ_{C}, 125 MHz, CDCl₃): 175.9, 172.3 (CO), 129.8 (×2) (CH═CH), 52.0 (NHCHCO), 42.0 (NCH₂), 36.6, 31.7 (×2), 29.7 (×2), 29.2 (×2), 29.1, 29.0, 28.8, 27.9, 27.2, 27.1, 25.6, 22.6 (CH₂) and 14.1 (CH₃). m/z (C₂₂H₄₀N₂O₂Na): 387.29700 (calculated: 387.2987).

### Example 10

### (S)-(Z)-3-(octadec-9-enoyl)amino-caprolactam

(S,S)-3-amino-caprolactam hydro-pyrrolidine-5-carboxylate (2 mmol) and Na₂CO₃ (6 mmol) in water (25 ml) were added to a solution of (Z)-octadec-9-enoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (514 mg; 66%).

Melting point: 66-67° C.

[α]_{D}²⁵ (c=1, CHCl₃)=+30.9.

IR: νₘₐₓ (cm⁻¹): 3327, 3268 (NH), 1655, 1631 (CO), 1523 (NH).

¹H NMR (δ_{H}, 500 M, CDCl₃): 6.88 (1H, d, J 5.5, CHNH), 6.74 (1H, br t, J 5, CH₂NH), 5.33-5.24 (2H, m, CH═CH), 4.49 (1H, ddd, J 11, 6, 1.5, CHNH), 3.29-3.14 (2H, m, CH₂NH), 2.16 (2H, t, J 7.5, CH₂CONH), 2.03 (1H, br d, J 13.5, ring CH), 1.99-1.89 (5H, m, ring CH+CH₂CH═CHCH₂), 1.84-1.72 (2H, m, ring CH), 1.58 (2H, br qn J 7.0, CH₂CH₂CONH), 1.42 (1H, br qd, J 14, 3, ring CH), 1.38-1.30 (1H, br m, ring CH), 1.30-1.14 (20H, m, (CH₂)₆CH₂CH═CHCH₂(CH₂)₄) and 0.83 (3H, t, J 7, CH₃).

¹³C NMR (δ_{C}, 125 MHz, CDCl₃): 175.9, 172.3 (CO), 129.9. 129.7 (CH═CH), 52.0 (NHCHCO), 42.0 (NCH₂), 36.6, 31.8, 31.7, 29.7 (×2), 29.5, 29.3 (×3), 29.2, 29.1, 28.8, 27.9, 27.2, 27.1, 25.6, 22.6 (CH₂) and 14.1 (CH₃).

m/z (C₂₄H₄₄N₂O₂Na): 415.32820 (calculated: 415.3300).

### Example 11

### (R)-(Z-3-(octadec-9-enoyl)amino-caprolactam

(R,R)-3-amino-caprolactam hydro-pyrrolidine-5-carboxylate (2 mmol) and Na₂CO₃ (6 mmol) in water (25 ml) were added to a solution of (Z)-octadec-9-enoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (574 mg; 73%).

Melting point: 66-67° C.

[α]_{D}²¹ (c=1, CHCl₃)=−31.4.

### Example 12

### (S)-3-(2′,2′-dimethyl-dodecanoyl)amino-caprolactam

(S,S)-3-amino-caprolactam hydro-pyrrolidine-5-carboxylate (2 mmol) and Na₂CO₃ (6 mmol) in water (25 ml) were added to a solution of 2,2-dimethyl-dodecanoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (543 mg; 80%).

Melting point: 41-42° C.

[α]_{D}²⁵ (c=1, CHCl₃)=+28.0.

IR: νₘₐₓ (cm⁻¹): 3403, 3265 (NH), 1673, 1641 (CO), 1497 (NH).

¹H NMR (δ_{H}, 500 MHz, CDCl₃): 7.08 (1H, d, J 5.5, CHNH), 6.67 (1H, br s, CH₂NH), 4.44 (1H, dd, J 11, 5.5, CHNH), 3.28-3.15 (2H, m, CH₂NH), 2.01 (1H, br d, J 13, ring CH), 1.98-1.89 (1H, m, ring CH), 1.84-1.72 (2H, m, ring CH), 1.47-1.30 (3H, br m, ring CH+CH₂CMe₂CONH), 1.27-1.15 (17H, br m, ring CH+(CH₂)₈) 1.13 (3H, s, CMeMe), 1.12 (3H, s, CMeMe) and 0.82 (3H, t, J 7, CH₂CH₃).

¹³C NMR (δ_{C}, 125 MHz, CDCl₃): 177.1, 176.0 (CO), 52.0 (NHCHCO), 41.9 (CMe₂), 42.1, 41.3, 31.8, 31.5, 30.1, 29.6, 29.5 (×2), 29.3, 28.9, 27.9 (CH₂), 25.3, 25.2 (CH₃), 24.8, 22.6 (CH₂) and 14.1 (CH₃).

m/z (C₂₀H₃₈N₂O₂Na): 361.28350 (calculated: 361.2831).

Compound 12 was later resynthesised on a larger scale, and this batch of material had the following properties: melting point 51-52° C. [α]_{D}²⁵ (c=1, CHCl₃)+28.0; [α]_{D}²⁵ (c=0.87, MeOH)+13.3.

### Example 13

### (S)-3-(decyloxycarbonyl)amino-caprolactam

(S,S)-3-amino-caprolactam hydro-pyrrolidine-5-carboxylate (2 mmol) and Na₂CO₃ (6 mmol) in water (25 ml) were added to a solution of decyl chloroformate (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (459 mg; 74%).

Melting point: 40-41° C.

[α]_{D}²⁵ (c=1, CHCl₃)=+31.4.

IR: νₘₐₓ (cm⁻): 3352, 3300 (NH), 1682, 1657, 1637 (CO), 1513 (NH).

¹H NMR (δ_{H}, 500 MHz, CDCl₃): 6.86 (1H, br s, CH₂NH), 6.72 (1H, d, J 6 CHNH), 4.49 (1H, dd, J 11, 6, CHNH), 3.99 (2H, t, J 6, OCH₂), 3.26-3.14 (2H, m, CH₂NH), 2.04 (1H, br d, J 13.5, ring CH), 2.00-1.91 (1H, m, ring CH), 1.82-1.68 (2H, m, ring CH), 1.55 (2H, br qn J 7.0, CH₂CH₂O), 1.48 (1H, br qd, J 14, 2.5, ring CH), 1.38-1.31 (1H, br m, ring CH), 1.29-1.17 (14H, m, (CH₂)₇) and 0.83 (3H, t, J 7, CH₃).

¹³C NMR (δ_{C}, 125 MHz, CDCl₃): 175.8, 155.9 (CO), 65.0 (OCH₂), 53.5 (NHCHCO), 42.0 (NCH₂), 32.1, 31.8, 29.5 (×2), 29.2 (×2), 29.0, 28.8, 28.0, 25.8, 22.6 (CH₂) and 14.1 (CH₃).

m/z (C₁₇H₃₂N₂O₃Na): 335.23190 (calculated: 335.2311).

### Example 14

### (S)-(E)-3-(dodec-2-enoyl)amino-caprolactam

(S,S)-3-amino-caprolactam hydro-pyrrolidine-5-carboxylate (2 mmol) and Na₂CO₃ (6 mmol) in water (25 ml) were added to a solution of dodec-2-enoyl chloride (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (472 mg; 77%).

Melting point: 87-88° C.

[α]_{D}²⁵ (c=1, CHCl₃)=+44.7.

IR: νₘₐₓ (cm⁻¹): 3382, 3331 (NH), 1660, 1616 (CO), 1520 (NH).

¹H NMR (δ_{H}, 500 MHz, CDCl₃): 6.94 (1H, d, J 5.5, CHNH), 6.84 (1H, br s, CH₂NH), 6.78 (1H, dt, J 15.5, 7, CH₂CH═CH), 5.80 (1H, d, J 15.5, CH₂CH═CH), 4.56 (1H, ddd, J 11, 6, 1.5, CHNH), 3.29-3.15 (2H, m, CH₂NH), 2.11 (2H, q, J 7, CH₂CH═CH), 2.07 (1H, br d, J 13.5, ring CH), 1.98-1.90 (1H, m, ring CH), 1.86-1.73 (2H, m, ring CH), 1.44 (1H, br qd, J 14, 2.5, ring CH), 1.41-1.29 (3H, br m, ring CH+CH₂CH₂CH═CH), 1.29-1.14 (12H, m, (CH₂)₆) and 0.82 (3H, t, J 6.5, CH₃).

¹³C NMR (δ_{C}, 125 MHz, CDCl₃): 175.9, 165.0 (CO), 144.8, 123.5 (CH═CH), 52.0 (NHCHCO), 42.0 (NCH₂), 32.0, 31.8, 31.6, 29.4 (×2), 29.2, 29.1, 28.8, 28.2, 27.9, 22.6 (CH₂) and 14.1 (CH₃).

m/z (C₁₈H₃₂N₂O₂Na): 331.23570 (calculated: 331.2361).

### Example 15

### (S)-3-(dec-9-enylaminocarbonyl)amino-caprolactam

(S,S)-3-amino-caprolactam hydro-pyrrolidine-5-carboxylate (2 mmol) and Na₂CO₃ (6 mmol) in water (25 ml) were added to a solution of dec-9-enyl isocyanate (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (347 mg; 56%).

Melting point: 98-99° C.

[α]_{D}²⁵ (c=1, CHCl₃)=+27.3.

IR: νₘₐₓ (cm⁻¹): 3365, 3327, 3276 (NH), 1619, (CO), 1551 (NH).

¹H NMR (δ_{H}, 500 MHz, CDCl₃): 6.64 (1H, br s, ring CH₂NH), 6.12 (1H, d, J 6 CHNH), 5.75 (1H, ddtd, J 17, 10, 6.5, 1.5, CH₂═CH), 5.21-5.12 (1H, br m, urea CH₂NH), 4.93 (1H, dq, J 17, 1.5, CHH═CH), 4.87 (1H, br d, J 10, CHH═CH), 4.49 (1H, dd, J 11, 6, NHCHCO), 3.25 (1H, ddd, J 15.5, 12, 4, ring CH₂N), 3.17 (1H, dt, J 14, 6, ring CH₂N), 3.11-3.02 (2H, m, urea NHCH₂), 2.05-1.87 (4H, br m, ring CH ×2+CH₂CH═CH), 1.82-1.70 (2H, m, ring CH), 1.48-1.36 (3H, br m, chain CH₂CH₂NH, + ring CH), 1.36-1.27 (3H, m, ring CH+chain CH₂) and 1.27-1.17 (8H, m, chain (CH₂)₄).

¹³C NMR (δ_{C}, 125 MHz, CDCl₃): 177.2, 157.6 (CO), 139.1, 114.1 (CH═CH), 52.7 (NHCHCO), 42.1, 40.3 (NCH₂), 33.7, 32.9, 30.3, 29.4, 29.3, 29.0, 28.8 (×2), 27.9 and 26.9 (CH₂).

m/z (C₁₇H₃₁N₃O₂Na): 332.23150 (calculated: 332.2314).

### Example 16

### (S)-3-(decylaminocarbonyl)amino-caprolactam

(S,S)-3-amino-caprolactam hydro-pyrrolidine-5-carboxylate (2 mmol) and Na₂CO₃ (6 mmol) in water (25 ml) were added to a solution of decyl isocyanate (2 mmol) in dichloromethane (25 ml) at ambient temperature and the reaction mixture was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (eluent: EtOAc to 9:1 EtOAc:MeOH) to give the title compound (401 mg, 64%).

Melting point: 97-98° C.

[α]_{D}²⁵ (c=1, CHCl₃)=+27.7.

IR: νₘₐₓ (cm⁻¹): 3359, 3316 (NH), 1621, (CO), 1558 (NH).

¹H NMR (δ_{H}, 500 MHz, CDCl₃): 6.62 (1H, br s, ring CH₂NH), 6.09 (1H, d, J 6 CHNH), 5.16 (1H, br t, J 5, urea CH₂NH), 4.48 (1H, ddd, J 11, 6, 1, NHCHCO), 3.26 (1H, ddd, J 16, 11, 5, ring CH₂N), 3.17 (1H, dt, J 15, 7, ring CH₂N), 3.11-3.02 (2H, m, urea NHCH₂), 2.02 (1H, br d J 14, ring CH), 1.96-1.87 (1H, m, ring CH), 1.83-1.70 (2H, m, ring CH), 1.48-1.27 (4H, br m, ring CH ×2+chain CH₂), 1.27-1.14 (14H, m, (CH₂)₇) and 0.82 (3H, t, J 7, CH₃).

¹³C NMR (δ_{C}, 125 MHz, CDCl₃): 177.2, 157.6 (CO), 52.7 (NHCHCO), 42.1, 40.4 (NCH₂), 32.9, 31.8, 30.2, 29.6, 29.5, 29.4, 29.3, 28.8, 27.9, 26.9, 22.6 (CH₂) and 14.1.

m/z (C₁₇H₃₃N₃O₂Na): 334.24880 (calculated: 334.2470).

### Example 17

### (R)-3-(2′,2′-Dimethyl-dodecanoyl)amino-caprolactam

(R,R)-3-amino-caprolactam hydro-pyrrolidine-5-carboxylate (2 mmol) and Na₂CO₃ (6 mmol) in water (25 ml) were added to a solution of 2,2-dimethyl-dodecanoyl chloride (2 mmol) in dichloromethane (25 ml) at ambient temperature and the reaction was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (EtOAc:hexanes 1:3 to EtOAc) to give (R)-3-(2′,2′-dimethyl-dodecanoyl)amino-caprolactam (515 mg, 76%); m.p. 48-49° C.; [α]_{D}²⁵ (c=1, CHCl₃) −25.7; [α]_{D}²⁵ (c=0.5, MeOH) −12.2.

Compound 17 was later resynthesised on a larger scale, and this batch of material had the following properties: melting point 50-51° C.

### Example 18

### (S)-3-(2′,2′-Dimethyl-pentanoyl)amino-caprolactam

(S,S)-3-amino-caprolactam hydro-pyrrolidine-5-carboxylate (20 mmol) and Na₂CO₃ (60 mmol) in water (50 ml) were added to a solution of 2,2-dimethyl-pentanoyl chloride (20 mmol) in dichloromethane (50 ml) at ambient temperature and the reaction was stirred for 12 hours. The organic layer was then separated and the aqueous phase was extracted with additional dichloromethane (2×25 ml). The combined organic layers were dried over Na₂CO₃ and reduced in vacuo. The residue was recrystallised from EtOAc/hexane to give (S)-3-(2′,2′-dimethyl-pentanoyl)amino-caprolactam (3.50 g, 77%); m.p. 84-85° C.; [α]_{D}²⁵ (c=1, CHCl₃) +30.7; νₘₐₓ/cm⁻¹ 3387, 3239 (NH), 1655, 1634 (CO), 1507 (NH); δ_{H} (500 MHz, CDCl₃) 7.08 (1H, d, J 5, CHNH), 6.53 (1H, br s, CH₂NH), 4.45 (1H, ddd, J 11, 5.5, 1.5, CHNH), 3.29-3.16 (2H, m, CH₂NH), 2.00 (1H, br d, J 13, ring CH), 1.98-1.92 (1H, m, ring CH), 1.84-1.73 (2H, m, ring CH), 1.47-1.30 (4H, br m, ring CH ×2+CH₂CM % CONH), 1.23-1.15 (2H, m, CH₂CH₃) 1.14 (3H, s, CMeMe), 1.13 (3H, s, CMeMe) and 0.84 (3H, t, J 7, CH₂CH₃); δ_{C} (125 MHz, CDCl₃) 177.0, 176.1 (CO), 52.1 (NHCHCO), 43.6, 42.0 (×2, one of which is CMe₂), 31.5, 28.9, 27.9 (CH₂), 25.3, 25.2 (CH₃), 18.0 (CH₂) and 14.5 (CH₃); m/z (M⁺ C₁₃H₂₄N₂O₂ requires 240.18378) 240.18437.

### Example 19

### (S)-3-(2′,2′-Dimethyl-pent-4-enoyl)amino-caprolactam

(S,S)-3-amino-caprolactam hydro-pyrrolidine-5-carboxylate (20 mmol) and Na₂CO₃ (60 mmol) in water (50 ml) were added to a solution of 2,2-dimethyl-pent-4-enoyl chloride (20 mmol) in dichloromethane (50 ml) at ambient temperature and the reaction was stirred for 2 hours. The organic layer was then separated and the aqueous phase was extracted with additional dichloromethane (2×25 ml). The combined organic layers were dried over Na₂CO₃ and reduced in vacuo. The residue was purified by silica column chromatography (1:1 EtOAc:hexane to EtOAc) to give (S)-3-(2′,2′-dimethyl-pent-4-enoyl)amino-caprolactam (1.43 g, 32%); m.p. 71-72° C.; [α]_{D}²⁵ (c=1, CHCl₃) +27.7; νₘₐₓ/cm⁻¹ 3395, 3304 (NH), 1675, 1633 (CO), 1534 (NH); δ_{H} (500 z, CDCl₃) 7.10 (1H, d, J 4.5, CHNH), 6.48 (1H, br s, CH₂NH), 5.68 (1H, ddt, J 17, 10, 7.5, CH═CH₂), 5.02 (1H, br d, J 17 CH═CHH), 5.00 (1H, br d, J 10, CH═CHH), 4.45 (1H, dd, J 11, 5.5, CHNH), 3.30-3.17 (2H, m, CH₂NH), 2.27 (1H, J 14, 7.5, CHHCH═CH₂), 2.22 (1H, dd, J 14, 7.5, CHHCH═CH₂), 2.01 (1H, br d, J 13, ring CH), 1.98-1.92 (1H, m, ring CH), 1.85-1.73 (2H, m, ring CH), 1.47-1.30 (2H, br m, ring CH ×2), 1.16 (3H, s, CMeMe) and 1.15 (3H, s, CMeMe); δ_{C} (125 MHz, CDCl₃) 176.4, 175.9 (CO), 134.2 (CH═CH₂), 117.8 (CH═CH₂), 52.1 (NHCHCO), 45.2, 42.1 (CH₂), 41.9 (CMe₂), 31.5, 28.9, 27.9 (CH₂), 25.0 and 24.9 (CH₃); m/z (M⁺ C₁₃H₂₂N₂O₂ requires 238.16813) 238.16834.

### Example 20

### (S)-3-(2′,2′-Dimethyl-propionyl)amino-caprolactam

(S,S)-3-amino-caprolactam hydro-pyrrolidine-5-carboxylate (5 mmol) and Na₂CO₃ (15 mmol) in water (15 ml) were added to a solution of 2,2-dimethyl-propionyl chloride

(5 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from EtOAc/hexane to give (S)-3-(2′,2′-dimethyl-propionyl)amino-caprolactam (645 mg, 61%); m.p. 126-127° C.; [α]_{D}²⁵ (c=1, CHCl₃) +39.5; νₘₐₓ/cm⁻¹ 3381, 3255 (NH, 1680, 1632 (CO), 1506 (NH); δ_{H} (500 MHz, CDCl₃) 7.10 (1H, d, J 5.0, CHNH), 6.75 (1H, br s, CH₂NH), 4.42 (1H, ddd, J 11, 5.5, 1.5, CHNH), 3.27-3.16 (2H, m, CH₂NH), 2.03-1.89 (2H, m, 2× ring CH), 1.83-1.71 (2H, m, 2× ring CH), 1.45-1.28 (2H, m, 2× ring CH) and 1.15 (9H, s, 3×CH₃); δ_{C} (125 MHz, CDCl₃) 177.7, 176.1 (CO), 52.1 (NHCHCO), 42.0 (CH₂N), 40.5 (CCO), 31.5, 28.9, 27.9 (CH₂ lactam), 27.4 (3×CH₃).

m/z (MNa⁺ C₁₁H₂₀N₂O₂Na requires 235.141699) 235.142237; (MH⁺ C₁₁H₂₁N₂O₂ requires 213.1597543) 213.160246.

### Example 21

### (S)-3-(2′,2′-Dimethyl-butyryl)amino-caprolactam

(S,S)-3-amino-caprolactam hydro-pyrrolidine-5-carboxylate (5 mmol) and Na₂CO₃ (15 mmol) in water (15 ml) were added to a solution of 2,2-dimethyl-butyryl chloride (5 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from EtOAc/hexane to give (S)-3-(2′,2′-dimethyl-propionyl)amino-caprolactam (562 mg, 50%); m.p. 106-107° C.; [α]_{D}²⁵ (c=1, CHCl₃) +33.6; νₘₐₓ/cm⁻¹ 3400, 3278 (NH), 1677, 1630 (CO), 1500 (NH); δ_{H} (500 MHz, CDCl₃) 7.08 (1H, d, J 5.0, CHNH), 6.72 (1H, br s, CH₂NH), 4.44 (1H, ddd, J 11, 5.5, 1.5, CHNH), 3.28-3.16 (2H, m, CH₂NH), 2.04-1.90 (2H, m, 2× ring CH), 1.83-1.72 (2H, m, 2× ring CH), 1.57-1.44 (2H, m, CH₂CH₃), 1.44-1.30 (2H, m, 2× ring CH) 1.12 (3H, s, CH₃) 1.11 (3H, s, CH₃) and 0.78 (3H, t, J 7.5, CH₂CH₃); δ_{C} (125 MHz, CDCl₃) 177.0, 176.0 (CO), 52.1 (NHCHCO), 42.2 (CCO), 42.0 (CH₂N), 33.7 (CH₂CH₃), 31.6, 28.9, 27.9 (CH₂ lactam), 24.8, 24.7 (CCH₃) and 9.1 (CH₂CH₃); m/z (MH⁺ C₁₂H₂₃N₂O₂ requires 227.1760) 227.1767.

### Example 22

### (S,E)-3-(2′,2′-Dimethyl-dodec-4′-enoyl)amino-caprolactam

(S,S)-3-amino-caprolactam hydro-pyrrolidine-5-carboxylate (10 mmol) and Na₂CO₃ (30 mmol) in water (30 ml) were added to a solution of 2,2-dimethyl-dodec-2-enoyl chloride (crude, from above reaction) (10 mmol) in dichloromethane (30 ml) at ambient temperature and the reaction was stirred for 12 hours. The organic layer was then separated and the aqueous phase was extracted with additional dichloromethane (2×25 ml). The combined organic layers were dried over Na₂CO₃ and reduced in vacuo. The residue was purified by silica column chromatography (1:1 EtOAc:hexanes to EtOAc) to give (S,E)-3-(2′,2′-dimethyl-dodec-4′-enoyl)amino-caprolactam as a colourless oil (2.12 g, 63%); [α]_{D}²⁵ (c=1, CHCl₃) +21.6; νₘₐₓ/cm⁻¹ 3264 (NH), 1639 (CO), 1497 (NH); δ_{H} (500 MHz, CDCl₃) 7.09 (1H, d, J 5.5, CHNH), 6.67-6.32 (1H, br m, CH₂NH), 5.42 (1H, dt, J 15, 6.5, CH═CH), 5.28 (1H, dt, J 15, 7, CH═CH), 4.44 (1H, dd, J 11, 5.5, CHNH), 3.30-3.17 (2H, m, CH₂NH), 2.20 (1H, dd, 13.5, 7, CH═CHCH₂), 2.14 (1H, dd, 13.5, 7, CH═CHCH₂), 2.01-1.87 (4H, br m, ring CH ×2, +CH₂CH═CH), 1.87-1.74 (2H, m, ring CH), 1.47-1.32 (2H, m, ring CH), 1.27-1.15 (10H, br m, (CH₂)₅) 1.13 (3H, s, CMeMe), 1.12 (3H, s, CMeMe) and 0.83 (3H, t, J 7, CH₂CH₃); δ_{C} (125 MHz, CDCl₃) 176.8, 176.0 (CO), 134.2, 125.2 (CH═CH), 52.1 (NHCHCO), 43.9 (CH₂), 42.1 (×2) (CH₂+CMe₂), 32.6, 31.8, 31.5, 30.1, 29.4, 29.1 (×2), 28.9, 27-9 (CH₂), 25.0, 24.8 (CH₃) and 22.6 (CH₃); m/z (MH⁺ C₂₀H₃₇N₂O₂ requires 337.2855) 337.2858.

### Example 23

### (S)-3-(2′,2′,5′-Trimethyl-hex-4′-enoyl)amino-caprolactam

(S,S)-3-amino-caprolactam hydro-pyrrolidine-5-carboxylate (4.11 g, 16 mmol) and Na₂CO₃ (5.09 g, 48 mmol) in water (50 ml) were added to a solution of 2,2,5-trimethyl-hex-4-enoyl chloride (16 mmol) in dichloromethane (50 ml) at ambient temperature and the reaction was stirred for 12 hours. The organic layer was then separated and the aqueous phase was extracted with additional dichloromethane (2×50 ml). The combined organic layers were dried over Na₂CO₃ and reduced in vacuo. The residue was purified by silica column chromatography (1:5 EtOAc:hexanes to EtOAc) to give (S)-3-(2′,2′,5′-trimethyl-hex-4′-enoyl)amino-caprolactam as a waxy solid (3.58 g, 84%); m.p. 43-44° C.; [α]_{D}²⁵ (c=1, CHCl₃) +23.2; νₘₐₓ/cm⁻¹ 3394, 3251 (NH), 1674, 1633 (CO), 1503 (NH); δ_{H} (500 MHz, CDCl₃) 7.11 (1H, d, J 5.0, CHNH), 6.65-6.45 (1H, br m, CH₂NH), 5.04 (1H, t, J 7.5, CH═C), 4.44 (1H, ddd, J 11, 5.5, 1.5, CHNH), 3.24-3.16 (2H, m, CH₂NH), 2.20 (1H, dd, J 14.5, 7.5, C═CHCH₂), 2.15 (1H, dd, J, 14.5, 7.5, C═CHCH₂), 2.03-1.90 (2H, m, 2× ring CH), 1.84-1.72 (2H, m, 2× ring CH), 1.65 (3H, s, CH₃), 1.56 (3H, s, CH₃), 1.45-1.28 (2H, m, 2× ring CH), 1.13 (3H, s, CH₃) and 1.12 (3H, s, CH₃); δ_{C} (125 MHz, CDCl₃) 176.9, 176.0 (CO), 134.1, 119.9 (CH═CH), 52.1 (NHCHCO), 42.5 (CH₂CMe₂), 42.1 (CH₂N), 39.0, 31.5, 28.9, 28.0 (CH, lactam), 26.0, 25.0, 24.9, 17.9 (CH₃); m/z (MH⁺ C₁₅H₂₇N₂O₂ requires 267.2073) 267.2063.

### Example 24

### (S)-3-(2′,2′,5′-Trimethyl-hexanoyl)amino-caprolactam

(S)-3-(2′,2′,5′-trimethyl-hex-4′-enoyl)amino-caprolactam (400 mg) was dissolved in EtOAc (25 ml), palladium hydroxide-on-carbon (20%, ca 100 mg) was added, and the mixture was stirred at ambient temperature under an atmosphere of hydrogen for 14 hours. The reaction was then filtered through a Celite® pad and the solvent was removed in vacuo to give (S)-3-(2′,2′,5′-trimethyl-hexanoyl)amino-caprolactam as a waxy solid (400 mg, 98%); m.p. 73-74° C.; [α]_{D}²⁵ (c=1, CHCl₃) +27.8; νₘₐₓ/cm⁻¹ 3249 (NH), 1654, 1638 (CO), 1502 (NH); 5H (500 MHz, CDCl₃) 7.08 (1H, d, J 5.0, CHNH), 6.75-6.55 (1H, br m, CH₂NH), 4.44 (1H, ddd, J 11, 5.5, 1.5, CHNH), 3.29-3.16 (2H, m, CH₂NH), 2.03-1.91 (2H, m, 2× ring CH), 1.84-1.73 (2H, m, 2× ring CH), 1.47-1.28 (5H, m, 2× ring CH+CH₂+CH(CH₃)₂), 1.13 (3H, s, CH₃), 1.12 (3H, s, CH₃), 1.08-1.02 (2H, m, CH₂), 0.82 (3H, s, CH₃), 0.80 (3H, s, CH₃); Bc (125 MHz, CDCl₃) 177.1, 176.1 (CO), 52.1 (NHCHCO), 42.1 (CH₂N), 41.9 (CH₂CMe₂), 39.0, 33.7, 31.5, 28.9 (CH₂), 28.4 (Me₂CH), 27.9 (CH₂), 25.3, 25.2, 22.6, 22.5 (CH₃); m/z (MH⁺ C₁₅H₂₉N₂O₂ requires 269.2229) 269.2219.

### Example 25

### (S)-3-(11′-bromo-undecanoyl)amino-caprolactam

(S)-3-amino-caprolactam hydrochloride (5 mmol) and Na₂CO₃ (15 mmol) in water (25 ml) were added to a solution of 11-bromo-undecanoyl chloride (5 mmol) in dichloromethane (25 ml) at ambient temperature and the reaction was stirred for 4 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give (S)-3-(11′-bromo-undecanoyl)amino-caprolactam (1.49 g, 79%); m.p. (EtOAc) 73-74° C.; [α]_{D}²⁵ (c=1, CHCl₃) +31.8; νₘₐₓ/cm⁻¹ 3342, 3287 (NH), 1668, 1634 (CO), 1515 (NH); δ_{H} (500 MHz, d₆-DMSO) 7.76 (1H, t, J 6.5, CH₂NH), 7.67 (1H, d, J 7, CHNH), 4.38 (1H, dd, J 11, 7, CHNH), 3.51 (2H, t, J 6.5, CH₂Br), 3.15 (1H, ddd, J 15.5, 10.5, 5, CHHNH), 3.05 (1H, dt, J 14, 7, CHHNH), 2.17-2.06 (2H, m, CH₂CONH), 1.85 (1H, dt, J 14, 3, C-5H), 1.82-1.68 (4H, m, C-4H, C-6H and CH₂CH₂Br), 1.62 (1H, qt, J 12, 3.5, C-5H), 1.46 (2H, br qn J 6.5, CH₂CH₂CONH), 1.41-1.31 (3H, m, C-4H and chain CH₂) and 1.31-1.13 (11H, m, (CH₂), +C-6H); δ_{C} (125 MHz, d₆-DMSO) 174.4 (CO-ring), 171.3 (CO-chain), 51.3 (NHCHCO), 40.7 (NCH₂), 35.3, 35.2, 32.4, 31.3, 29.0, 28.9 (×3), 28.7, 28.2, 27.8, 27.6 and 25.4 (CH₂); m/z (MH⁺ BrC₁₇H₃₂N₂O₂ requires 375.1647) 375.1655.

### Example 26

### (S)-3-(11′-azido-undecanoyl)amino-caprolactam

Sodium azide (650 mg, 10 mmol) was added to (S)-3-(11-bromo-undecanoyl)amino-caprolactam (375 mg, 1 mmol) in DMF (2 ml) and the mixture was heated at 60° C. for 14 hours. The solvent was then removed in vacuo and the residue was partitioned between water (20 ml) and EtOAc (3×20 ml). The combined organic layers were washed with 1M HCl_{aq} (2×20 ml) and then dried over Na₂CO₃ and reduced in vacuo. The residue was purified by recrystallisation from EtOAc to give (S)-3-(11′-azido-undecanoyl)amino-caprolactam (221 mg, 66%); m.p. (EtOAc) 71-72° C.; [α]_{D}²⁵ (c=1, CHCl₃) +34.7; νₘₐₓ/cm⁻¹ 3344, 3289 (NH), 2101 (N3) 1668, 1631 (CO), 1516 (NH); δ_{H} (500 MHz, d-DMSO) 7.77 (1H, t, J 6, CH₂NH), 7.67 (1H, d, J 7, CHNH), 4.38 (1H, dd, J 11, 7, CHNH), 3.30 (2H, t, J 7, CH₂N₃), 3.15 (1H, ddd, J 15.5, 10.5, 5, CHHNH), 3.05 (1H, dt, J 14, 5.5, CHHNH), 2.17-2.07 (2H, m, CH₂CONH), 1.85 (1H, dt, J 14, 3.5, C-5H), 1.82-1.68 (2H, m, C-4H, C-6H), 1.62 (1H, qt, J 13, 3.5, C-5H), 1.51 (4H, m, CH₂CH₂CONH and CH₂CH₂N₃), 1.36 (1H, qd, J 13, 3, C-4H), and 1.33-1.13 (13H, m, (CH₂)₆+C-6H); δ_{C} (125 MHz, d₆-DMSO) 174.4 (CO-ring), 171.3 (CO-chain), 51.3 (NHCHCO), 50.7 (CH₂N₃), 40.7 (NCH₂), 35.3, 31.3, 29.0 (×2), 28.9, 28.7, 28.6, 28.3, 27.8, 26.2 and 25.4 (CH₂); m/z (MNa⁺ C₁₇H₃₁N₅O₂Na requires 360.2375) 360.2360.

### Example 27

### (S) Sodium 3-(undecanoyl)amino-caprolactam 11′-sulfonate tetrahydrate

sodium sulfite (630 mg, 5 mmol) in water (3 ml) was added to (S)-3-(11-bromo-undecanoyl)amino-caprolactam (375 mg, 1 mmol) in ethanol (2 ml) and the mixture was heated at reflux for 14 hours. The cooled reaction mixture was then added to ethanol (25 ml) and the reaction was filtered. The solvent was then removed in vacuo to give (S) sodium 3-(undecanoyl)amino-caprolactam 11′-sulfonate tetrahydrate (456 mg, 97%); m.p. (EtOAc) 208-210° C.; [α]_{D}²⁵ (c=1, H₂O) −15.5; νₘₐₓ/cm⁻¹ 3430, 3344, 3289 (NH+H₂O), 1667, 1643 (CO), 1530 (NH) 1195, 1183 (SO₃, asymm.), 1064 (SO₃, symm.); δ_{H} (500 MHz, d₆-DMSO) 7.76 (1H, t, J 6, CH₂NH), 7.70 (1H, d, J 7, CHNH), 4.35 (1H, dd, J 10, 7.5, CHNH), 3.42 (8H, s, 4×H₂O) 3.17-3.00 (2H, m, CH₂NH), 2.47-2.38 (2H, m, CH₂SO₃), 2.17-2.05 (2H, m, CH₂CONH), 1.82 (1H, br s, J 13.5, C-5H), 1.75-1.66 (2H, m, C-4H, C-6H), 1.65-1.50 (3H, m, C-5H+chain CH₂), 1.47-1.40 (2H, m, chain CH₂) 1.35 (1H, qd, J 13, 3, C-4H), and 1.30-1.11 (13H, m, (CH₂)₆+C-6H); δ_{C} (125 MHz, d₆-DMSO) 174.5 (CO-ring), 171.5 (CO-chain), 51.6 (CH₂SO₃), 51.4 (NHCHCO), 40.8 (NCH₂), 35.3, 31.3, 29.1 (×3), 29.0 (×2), 28.8, 28.6, 27.8, 25.5 and 25.1 (CH₂); m/z (MNa⁺ C₁₇H₃₁N₂O₅SNa₂ requires 421.1749) 421.1748.

### Example 28

### (S)-3-(Decanesulfonyl)amino-caprolactam

(S)-3-amino-caprolactam hydrochloride (3 mmol) and Na₂CO₃ (9 mmol) in water (20 ml) were added to a solution of decanesulfonylchloride (3 mmol) in dichloromethane (20 ml) at ambient temperature and the reaction was stirred for 10 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc/hexanes to give (S)-3-(decanesulfonyl)amino-caprolactam (481 mg, 48%); m.p. 98-99° C.; [α]_{D}²⁵ (c=1, MeOH) +22.7; νₘₐₓ/cm⁻¹ 3365, 3248 (NH), 1657 (CO), 1324, 1142 (SO₂N); δ_{H} (500 MHz, CDCl₃) 6.35-6.18 (1H, m, CH₂NH), 5.71 (1H, d, J 6, CHNH), 4.11 (1H, ddd, J 11.5, 6, 2, CHNH), 3.31-3.18 (2H, m, CH₂NH), 2.98-2.92 (2H, m, CH₂SO₂), 2.09 (1H, br d, J 14, ring CH), 2.06-1.97 (1H, m, ring CH), 1.88-1.59 (5H, m, CH₂CH₂SO₂+3 ring CH), 1.43-1.33 (3H, m, chain CH₂+ ring CH), 1.32-1.18 (12H, m, CH₃(CH₂)₆) and 0.86 (3H, m, CH₃); δ_{C} (125 MHz, CDCl₃) 174.8 (CO) 55.5 (NHCHCO), 53.5 (CH₂SO₂), 40.7 (NCH₂), 33.9, 31.8, 29.4, 29.3, 29.2, 29.1, 28.6, 28.3, 27.9, 23.5, 22.6 (CH₂), and 14.1 (CH₃); m/z (MNa⁺ C₁₆H₃₂N₂O₃SNa requires 355.2031) 355.2054; anal (C₁₆H₃₂N₂O₃S requires C, 57.8; H, 9.7; N, 8.4) C, 57.8; H, 9.7; N, 8.3.

### Example 29

### (S)-3-(Dodecanesulfonyl)amino-caprolactam

(S)-3-amino-caprolactam hydrochloride (2 mmol) and Na₂CO₃ (6 mmol) in water (20 ml) were added to a solution of dodecanesulfonylchloride (2 mmol) in dichloromethane (20 ml) at ambient temperature and the reaction was stirred for 10 hours. The organic layer was then separated and the aqueous phase was extracted with additional dichloromethane (2×25 ml). The combined organic layers were dried over Na₂CO₃ and reduced in vacuo. The residue was purified by silica column chromatography (hexanes:EtOAc 3:1 to 100% EtOAc) and then by recrystallisation from heptane to give (S)-3-(dodecanesulfonyl)amino-caprolactam (302 mg, 42%); m.p. 100-101° C.; [α]_{D}²⁵ (c=1, MeOH) +22.4; νₘₐₓ/cm⁻¹ 3366, 3247 (NH), 1657 (CO), 1324, 1143 (SO₂N); δ_{H} (500 MHz, CDCl₃) 6.66 (1H, t, J 6, CH₂NH), 5.78 (1H, d, J 6, CHNH), 4.10 (1H, ddd, J 11, 6, 2, CHNH), 3.29-3.17 (2H, m, CH₂NH), 2.97-2.90 (2H, m, CH₂SO₂), 2.12-2.03 (1H, m, ring CH), 2.03-1.96 (1H, m, ring CH), 1.88-1.59 (5H, m, CH₂CH₂SO₂+3 ring CH), 1.43-1.32 (3H, m, ring CH+chain CH), 1.32-1.18 (16H, m) and 0.85 (3H, m, CH₃); δ_{C} (125 MHz, CDCl₃) 175.0 (CO) 55.5 (NHCHCO), 53.5 (CH₂SO₂), 42.1 (NCH₂), 33.8, 31.8, 29.6 (×2), 29.5, 29.3 (×2), 29.1, 28.6, 28.3, 27.9, 23.5, 22.6 (CH₂), and 14.1 (CH₃); m/z (MNa⁺ C₁₈H₃₆N₂O₃SNa requires 383.2339) 383.2351; anal (C₁₈H₃₆N₂O₃S requires C, 60.0; H, 10.1; N, 7.8) C, 59.9; H, 10.2; N, 7.7.

### Example 30

### (S)-3-(Tetradecanesulfonyl)amino-caprolactam

(S)-3-amino-caprolactam hydrochloride (2 mmol) and Na₂CO₃ (6 mmol) in water (20 ml) were added to a solution of tetradecanesulfonylchloride (2 mmol) in dichloromethane (20 ml) at ambient temperature and the reaction was stirred for 10 hours. The organic layer was then separated and the aqueous phase was extracted with additional dichloromethane (2×25 ml). The combined organic layers were dried over Na₂CO₃ and reduced in vacuo. The residue was purified by silica column chromatography (hexanes:EtOAc 3:1 to 100% EtOAc) and then by recrystallisation from heptane to give (S)-3-(tetradecanesulfonyl)amino-caprolactam (373 mg, 48%); m.p. 100-101° C.; [α]_{D}²⁵ (c=1, CHCl₃) +14.4; νₘₐₓ/cm⁻¹ 3361, 3250 (NH), 1658 (CO), 1324, 1140 (SO₂N); δ_{H} (500 MHz, CDCl₃) 6.64 (1H, t, J 6, CH₂NH), 5.74 (1H, d, J 6, CHNH), 4.11 (1H, ddd, J 11.5, 6, 2, CHNH), 3.30-3.17 (2H, m, CH₂NH), 2.97-2.92 (2H, m, CH₂SO₂), 2.12-2.05 (1H, m, ring CH), 2.05-1.96 (1H, m, ring CH), 1.87-1.59 (5H, m, CH₂CH₂SO₂+3 ring CH), 1.42-1.32 (3H, m, ring CH+chain CH₂), 1.32-1.18 (20H, m, chain CH₂) and 0.86 (3H, m, CH₃); δ_{C} (125 MHz, CDCl₃) 174.9 (CO) 55.5 (NHCHCO), 53.4 (CH₂SO₂), 42.2 (NCH₂), 33.8, 31.9, 29.6 (×4), 29.5, 29.3 (×2), 29.1, 28.6, 28.3, 27.9, 23.5, 22.7 (CH₂), and 14.1 (CH₃); m/z (MNa⁺ C₂₀H₄₀N₂O₃SNa requires 411.2652) 411.2655; anal (C₂₀H₄₀N₂O₃S requires C, 61.8; H, 10.4; N, 7.2) C, 61.9; H, 10.5; N, 7.2.

### Example 31

### (S)-3-(Hexadecanesulfonyl)amino-caprolactam

(S)-3-amino-caprolactam hydrochloride (2 mmol) and Na₂CO₃ (6 mmol) in water (20 ml) were added to a solution of hexadecanesulfonylchloride (2 mmol) in dichloromethane (20 ml) at ambient temperature and the reaction was stirred for 10 hours. The organic layer was then separated and the aqueous phase was extracted with additional dichloromethane (2×25 ml). The combined organic layers were dried over Na₂CO₃ and reduced in vacuo. The residue was purified by silica column chromatography (hexanes:EtOAc 3:1 to 100% EtOAc) and then by recrystallisation from heptane to give (S)-3-(hexadecanesulfonyl)amino-caprolactam (553 mg, 66%); m.p. 100-101° C.; [α]_{D}²⁵ (c=1, CHCl₃) +14.1; νₘₐₓ/cm⁻¹ 3356, 3249 (NH), 1659 (CO), 1323, 1140 (SO₂N); δ_{H} (500 MHz, CDCl₃) 6.55 (1H, t, J 6, CH₂NH), 5.76 (1H, d, J 6, CHNH), 4.11 (1H, ddd, J 11.5, 6, 2, CHNH), 3.30-3.17 (2H, m, CH₂NH), 2.94 (2H, t, J 8, CH₂SO₂), 2.12-2.04 (1H, m, ring CH), 2.04-1.97 (1H, m, ring CH), 1.87-1.58 (5H, m, CH₂CH₂SO₂+3 ring CH), 1.42-1.32 (3H, m, ring CH+chain CH₂), 1.32-1.18 (24H, m, chain CH) and 0.86 (3H, m, CH₃); δ_{C} (125 MHz, CDCl₃) 174.9 (CO) 55.5 (NHCHCO), 53.5 (CH₂SO₂), 42.1 (NCH₂), 33.8, 31.9, 29.7 (×2), 29.6 (×4), 29.5, 29.3 (×2), 29.1, 28.6, 28.3, 27.9, 23.5, 22.7 (CH₂), and 14.1 (CH₃); m/z (MNa⁺ C₂₀H₄₀N₂O₃SNa requires 439.2965) 439.2980; anal (C₂₂H₄₄N₂O₃S requires C, 63.4; H, 10.6; N, 6.7) C, 63.1; H, 10.6; N, 6.6.

### Example 32

### (S)-3-(Octadecanesulfonyl)amino-caprolactam

(S)-3-amino-caprolactam hydrochloride (2 mmol) and Na₂CO₃ (6 mmol) in water (20 ml) were added to a solution of octadecanesulfonylchloride (2 mmol) in dichloromethane (20 ml) at ambient temperature and the reaction was stirred for 10 hours. The organic layer was then separated and the aqueous phase was extracted with additional dichloromethane (2×25 ml). The combined organic layers were dried over Na₂CO₃ and reduced in vacuo. The residue was purified by silica column chromatography (hexanes:EtOAc 3:1 to 100% EtOAc) and then by recrystallisation from heptane to give (S)-3-(octadecanesulfonyl)amino-caprolactam (545 mg, 61%); m.p. 99-100° C.; νₘₐₓ/cm⁻¹ 3356, 3249 (NH), 1659 (CO), 1323, 1140 (SO₂N); δ_{H} (500 MHz, CDCl₃) 6.15 (1H, t, J 6, CH₂NH), 5.69 (1H, d, J 6, CHNH), 4.12 (1H, ddd, J 11.5, 6, 2, CHNH), 3.30-3.18 (2H, m, CH₂NH), 2.97-2.92 (2H, m, CH₂SO₂), 2.12-2.07 (1H, m, ring CH), 2.06-1.97 (1H, m, ring CH), 1.87-1.56 (5H, m, CH₂CH₂SO₂+3 ring CH), 1.42-1.32 (3H, m, ring CH+chain CH₂), 1.32-1.18 (28H, m, chain CH₂) and 0.86 (3H, m, CH₃); m/z (MNa⁺ C₂₄H₄₈N₂O₃SNa requires 467.3277852) 467.330047.

### Example 33

### (S)-aminocaprolactam-Glycine-(L)-N(Boc)-Tryptophan

This tripeptide was made on a solid-phase automated peptide synthesiser using (S)-aminocaprolactam for the final peptide coupling step. Mr(Calc)=471.5110. Observed Mr by mass spectrometry 471.6. Purity (% TIC in molecular ion peak)=90%

### Example 34

### (S)-aminocaprolactam-(L)-valine-(L)-Desaminotryptophan

This tripeptide was made on a solid-phase automated peptide synthesiser using (S)-aminocaprolactam for the final peptide coupling step. Mr(Calc)=398.4600. Observed Mr by mass spectrometry 398.3. Purity (% TIC in molecular ion peak)=96%

Examples 35-38 of Intermediate Compounds useful in the synthesis of compounds of the invention:

### Example 35

### Intermediate

### (E)-Methyl 2,2-dimethyl-dodec-4-enoate

butyllithium (3.8 M, 10 mmol) was added to a solution of diisopropylamine (1.42 ml, 10 mmol) in dry THF at −78° C. under N₂. The reaction was stirred at −78° C. for 20 minutes and then methyl isobutyrate (1.15 ml, 10 mmol) was added. The reaction was stirred at −78° C. for 1 hour, and then (E)-dec-2-enyl bromide (2.19 g, 10 mmol) was added and the reaction was allowed to warm to ambient temperature over 14 hours. The reaction solvent was then removed in vacuo, and the residue was partitioned between pH 2 aqueous buffer (0.5 M NaHSO₄/0.5 M Na₂SO₄) (100 ml) and hexane (3×100 ml). The combined organic layers were dried over Na₂SO₄ and the hexane solvent removed in vacuo to give crude (E)-methyl 2,2-dimethyl-dodec-4-enoate (>90% pure) (2.27 g) as a colourless oil; νₘₐₓ/cm⁻¹ 1734 (CO); δ_{H} (400 MHz, CDCl₃) 5.42 (1H, br dt, J 15, 6.5, CH═CH), 5.30 (1H, dtt, J 15, 7, 1, CH═CH), 3.64 (3H, s, OCH₃), 2.18 (2H, dd, J 7, 1, CH₂CMe₂), 1.96 (2H, br q, J 6.5, CH₂CH₂CH═CH), 1.35-1.20 (10H, m, (CH₂)₅CH₃), 1.14 (6H, s, C(CH₃)₂), 0.87 (3H, t, J 6.5, CH₂CH₃); δ_{C} (125 MHz, CDCl₃) 178.2 (CO), 134.1, 125.2 (HC═CH), 51.5 (OCH₃), 43.6 (CH₂), 42.6 (Me₂CCO), 32.6, 31.8, 29.5, 29.1, 29.0 (CH₂), 24.7 (C(CH₃)×2), 22.6 (CH₂), 14.1 (CH₂CH₃); m/z (MH⁺ C₁₅H₂₉N₂O₂ requires 241.2168) 241.2169.

### Example 36

### Intermediate

### (E)-2,2-Dimethyl-dodec-4-enoyl chloride

the entire product from the above reaction was then dissolved in ethanol (50 ml) and added to a solution of NaOH (2.0 g, 50 mmol) in water (25 ml). The mixture was heated at reflux for 6 hours, allowed to cool and the solvents were then removed in vacuo. The residue was partitioned between pH 2 aqueous buffer (0.5 M NaHSO₄/0.5 M Na₂SO₄) (100 ml) and diethyl ether (3×100 ml). The combined organic layers were dried over Na₂SO₄ and the ether solvent removed in vacuo to give crude (E)-2,2-dimethyl-dodec-4-enoic acid (>90% pure) as a colourless oil; δ_{H} (400 MHz, CDCl₃) 5.46 (1H, br dt, J 15, 6.5, CH═CH), 5.35 (1H, dtt, J 15, 7, 1, CH═CH), 2.22 (2H, dd, J 7, 1, CH₂CMe₂), 1.98 (2H, br q, J 6.5, CH₂CH₂CH═CH), 1.37-1.21 (10H, m, (CH₂)₅CH₃), 1.17 (6H, s, C(CH₃)₂), 0.87 (3H, t, J 6.5, CH₂CH₃). The crude acid was dissolved in dichloromethane (50 ml) and oxalyl chloride (3 ml) was added along with a drop of DMF. The reaction was stirred for 1 hour and the solvent was removed in vacuo to give crude (E)-2,2-dimethyl-dodec-4-enoyl chloride which was all used without purification in the next step.

### Example 37

### Intermediate

### Methyl 2,2,5-trimethyl-hex-4-enoate

butyllithium (2.9 M, 50 mmol) was added to a solution of diisopropylamine (7.2 ml, 50 mmol) in dry THF (200 ml) at −78° C. under N₂. The reaction was stirred at −78° C. for 20 minutes and then methyl isobutyrate (5.7 ml, 50 mmol) was added. The reaction was stirred at −78° C. for 1 hour, and then 3-methyl-but-2-enyl bromide (5.8 ml, 50 mmol) was added and the reaction was allowed to warm to ambient temperature over 14 hours. The reaction solvent was then removed in vacuo, and the residue was partitioned between pH 2 aqueous buffer (0.5 M NaHSO₄/0.5 M Na₂SO₄) and hexane (3×250 ml). The combined organic layers were dried over Na₂SO₄ and the hexane solvent removed in vacuo to give methyl 2,2,5-trimethyl-hex-4-enoate as a colourless oil (6.93 g 81%); νₘₐₓ/cm⁻¹ 1732 (CO); 5H (400 MHz, CDCl₃) 5.04 (1H, tsept, J 7.5, 1.5, CH═C), 3.63 (3H, s, OCH₃), 2.20 (2H, d, J 7.5, CHCH₂), 1.68 (3H, br s, CH═CMeMe), 1.58 (3H, br s, CH═CMeMe), 1.14 (6H, s, (CH₃)₂CO); δ_{C} (125 MHz, CDCl₃) 178.4 (CO), 134.1 (Me₂C═CH), 119.8 (Me₂C═CH), 51.6 (OCH₃), 42.8 (Me₂CCO), 38.7 (CH₂), 25.9, 24.7 (×2), 17.8 (CCH₃); m/z (MH⁺ C₁₀H₁₉O₂ requires 171.1385) 171.1388.

### Example 38

### Intermediate

### 2,2,5-Trimethyl-hex-4-enoyl chloride

methyl 2,2,5-trimethyl-hex-4-enoate (2.74 g, 16 mmol) was dissolved in ethanol (50 ml) and added to a solution of NaOH (3.0 g, 75 mmol) in water (35 ml). The mixture was heated at reflux for 6 hours, allowed to cool and the solvents were then removed in vacuo. The residue was partitioned between pH 2 aqueous buffer (0.5 M NaHSO₄/0.5 M Na₂SO₄) and diethyl ether (3×150 ml). The combined organic layers were dried over Na₂SO₄ and the ether solvent removed in vacuo to give crude 2,2,5-trimethyl-hex-4-enoic acid (>95% pure) as a colourless oil; δ_{H} (400 MHz, CDCl₃) 5.12 (1H, tsept, J 7.5, 1.5, CH═C), 2.25 (2H, d, J 7.5, CHCH₂), 1.71 (3H, br s, CH═CMeMe), 1.60 (3H, br s, CH═CMeMe), 1.18 (6H, s, (CH₃)₂CO). The crude acid was dissolved in dichloromethane (50 ml) and oxalyl chloride (3 ml) was added along with a drop of DMF. The reaction was stirred for 1 hour and the solvent was removed in vacuo to give crude 2,2,5-trimethyl-hex-4-enoyl chloride which was all used without purification in the next step.

### Example 39

This compound has two head groups on either side of a 2,2,6,6 tetramethyl heptanoic acid. It is in effect a dimer of the corresponding 2,2-dimethyl compound of the invention:

### (S,S) N,N-bis-(2′-oxo-azepan-3′-yl) 2,2,6,6-tetramethylheptadiamide

(S,S)-3-amino-caprolactam hydro-pyrrolidine-5-carboxylate (2 mmol) and Na₂CO₃ (6 mmol) in water (25 ml) were added to a solution of 2,2,6,6-tetramethyl-heptandioyl dichloride (1 mmol) in dichloromethane (25 ml) at ambient temperature and the reaction was stirred for 2 hours. The organic layer was then separated and the aqueous phase was extracted with additional dichloromethane (2×25 ml). The combined organic layers were dried over Na₂CO₃ and reduced in vacuo. The residue was purified by recrystallisation from EtOAc to give (S,S)-dimer (199 mg, 46%); m.p. 234-236° C.; [α]_{D}²⁵ (c=1, CHCl₃) +29.4; νₘₐₓ/cm⁻¹ 3379, 3255 (NH), 1683, 1637 (CO), 1507, 1497 (NH); δ_{H} (500 MHz, CDCl₃) 7.07 (2H, d, J 5.5, CHNH), 6.42 (2H, br s, CH₂NH), 4.44 (2H, ddd, J 11, 5.5, 1.5, CHNH), 3.31-3.17 (4H, m, CH₂NH), 2.04-1.94 (4H, m, ring CH), 1.86-1.73 (4H, m, ring CH), 1.51-1.31 (8H, br m, 2× ring CH+CH₂CMe₂) and 1.12 (14H, m, chain CH₂CH₂CH₂+CMe₂); δ_{C} (125 MHz, CDCl₃) 176.9, 175.9 (CO), 52.1 (NHCH), 42.0 (CMe₂), 42.1, 41.5, 31.5, 28.9, 28.0 (CH₂), 25.3, 25.1 (CH₃) and 20.0 (CH₂); m/z (M⁺ C₂₃H₄₀N₄O₄ requires 436.30496) 436.30437.

### Example 40

### (S)-3-(1′,1′-dimethylundecanesulfonyl)amino-caprolactam

This compound is the sulfonamide analogue of Example 12.

### Example 41

### (S)-3-(2′-Propylpentanoyl)amino-caprolactam

(S,S)-3-amino-caprolactam hydro-pyrrolidine-5-carboxylate (5 mmol) and Na₂CO₃ (15 mmol) in water (15 ml) were added to a solution of 2-propylpentanoyl chloride (5 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from hexane to give (S)-3-(2′-propylpentanoyl)amino-caprolactam (1.02 g, 80%); m.p. (hexanes) 114-118° C.; [α]_{D}²⁵ (c=1, CHCl₃) +29.4; νₘₐₓ/cm⁻¹ 3303 (NH), 1686, 1633 (CO), 1537 (NH); δ_{H} (500 MHz, CDCl₃) 6.88 (1H, d, J 5.5, CHNH), 6.52 (1H, br s, CH₂NH), 4.52 (1H, ddd, J 11, 6, 1.5, CHNH), 3.30-3.16 (2H, m, CH₂NH), 2.13-2.02 (2H, m, (CH₂)₂CHCO and lactam ring CH), 2.02-1.92 (1H, m, lactam ring CH), 1.86-1.74 (2H, m, lactam ring CH ×2), 1.57-1.50 (2H, m, sidechain CH₂), 1.42 (1H, br qd, J 13.5, 3.5, lactam ring CH), 1.38-1.29 (2H, m, lactam ring CH+ side chain CH₂), 1.29-1.19 (4H, m, sidechain CH ×4), 0.85 (3H, t, J 7.5, CH₃) and 0.84 (3H, t, J 7.5, CH₃); δ_{C} (125 MHz, CDCl₃) 175.8, 175.2 (CO), 51.9 (NHCHCO), 47.2 (CH), 42.1, 35.3, 35.1, 31.7, 28.9, 27.9, 20.7 (×2) (CH₂) and 14.1 (×2) (CH₃); m/z (MH⁺ C₁₄H₂₇N₂O₂ requires 255.2073) 255.2083.

### Example 42(a)

### (3S,2′R) and

### Example 42(b)

### (3S,2′S)-3-(2′-Ethylhexanoyl)amino-caprolactam

(S,S)-3-amino-caprolactam hydro-pyrrolidine-5-carboxylate (5 mmol) and Na₂CO₃ (15 mmol) in water (15 ml) were added to a solution of (+/−) 2-ethylhexanoyl chloride (5 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from hexane to give a mixture of (3S,2′R) and (3S,2′S)-3-(2′-ethylhexanoyl)amino-caprolactam (328 mg, 26%); νₘₐₓ/cm⁻¹ 3306 (NH), 1686, 1633 (CO), 1537 (NH); δ_{H} (500 MHz, CDCl₃) 6.89 (2H, d, J 5, CHNH, both isomers), 6.53 (2H, br s, CH₂NH, both isomers), 4.52 (2H, ddd, J 11, 6, 1.5, CHNH, both isomers), 3.30-3.16 (4H, m, CH₂NH, both isomers), 2.06 (2H, br d, J 13.5, lactam CH ×2, both isomers), 2.02-1.92 (4H, m, (CH₂)₂CHCO ×2 and lactam ring CH ×2, both isomers), 1.86-1.74 (4H, m, lactam ring CH ×4, both isomers), 1.63-1.50 (4H, m, sidechain CH₂), 1.50-1.30 (8H, m, lactam ring CH ×4+ sidechain CH₂×₄, both isomers), 1.30-1.14 (8H, m, side chain CH₂×₈, both isomers), 0.85 (3H, t, J 7.5, CH₃, one isomer) and 0.82 (3H, t, J 7.5, CH₃, one isomer); δ_{C} (125 MHz, CDCl₃) 175.8, 175.1 (CO), 52.0, 51.9 (NHCHCO), 49.3 (×2) (CH), 42.0 (×2), 32.5, 32.3, 31.7 (×2), 29.7 (×2), 28.8 (×2), 27.9 (×2), 26.1, 25.9, 22.7 (×2), 14.0, 13.9 (CH₃) and 12.0 (×2) (CH₃); m/z (N C₁₄H₂₆N₂O₂ requires 254.1994) 254.1995.

### Example 43

### 3,3-Dimethyldodecanoic acid (Intermediate)

CuI (2 mmol), trimethylsilyl chloride (24 mmol) and methyl 3,3-dimethylacrylate (20 mmol) in THF (25 mmol) was cooled to −15° C., and a solution of nonylmagnesium bromide (24 mmol) in THF (80 ml) was added over one hour. The reaction was allowed to warm to room temperature overnight and it was then quenched by the addition of saturated aqueous ammonium chloride. The THF was removed in vacuo and the residue was partitioned between hexanes and water. The organic layer was reduced in vacuo and the crude methyl 3,3-dimethyldodecanoate was dissolved in ethanol (50 ml). KOH (100 mmol) in water (100 ml) was added and the reaction was heated at reflux for 18 hours. The reaction was then allowed to cool, and the solvent was removed in vacuo. and the residue was partitioned between hexane and water. The aqueous layer was then acidified to pH 2 with aqueous HCl. and extracted with diethyl ether. The ether layer was dried over Na₂SO₄ and the solution was then reduced in vacuo to give 3,3-dimethyldodecanoic acid as an oil (3.47 g, 76%); νₘₐₓ/cm⁻¹ 1702 (CO); δ_{H} (500 MHz, CDCl₃) 11.12 (1H, br s, OH), 2.21 (2H, s, CH₂CO); 1.32-1.20 (16H, m, (CH₂)₈), 1.00 (6H, s, C(CH₃)₂) and 0.87 (3H, t, J 7, CH₂CH₃); δ_{C} (125 MHz, CDCl₃) 179.1 (CO), 45.9, 42.3 (CH₂), 33.2 (C(CH₃)₂), 31.9, 30.3, 29.6 (×2), 29.3, 27.1 (×2) (C(CH₃)₂), 24.0, 22.6 (CH₂) and 14.1 (CH₃); m/z (M⁺ C₁₄H₂₈O₂ requires 228.2089) 228.2082.

### Example 44

### 3,3-Dimethyldodecanoyl chloride (Intermediate)

3,3-dimethyldodecanoic acid (5 mmol) was dissolved in CH₂Cl₂ (20 ml) oxalyl chloride (1 ml) and dimethyl formamide (1 drop) was added. After 1 hour the reaction was reduced in vacuo to give crude 3,3-dimethyldodecanoyl chloride which was used directly in the synthesis of (S)-3-(3′,3′-dimethyldodecanoyl)amino-caprolactam.

### Example 45

### (S)-3-(3′,3′-Dimethyldodecanoyl)amino-caprolactam

(S,S)-3-amino-caprolactam hydro-pyrrolidine-5-carboxylate 2 (5 mmol) and Na₂CO₃ (15 mmol) in water (15 ml) were added to a solution of 3,3-dimethyldodecanoyl chloride (5 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from hexane to give (S)-3-(3′,3′-dimethyldodecanoyl)amino-caprolactam (1.14 g, 68%); m.p. (hexanes) 123-125° C.; [α]_{D}²⁵ (c=1, CHCl₃) +28.6; νₘₐₓ/cm⁻¹ 3279 (NH), 1646 (CO), 1498 (NH); δ_{H} (500 MHz, CDCl₃) 6.81 (1H, d, J 5.5, CHNH), 6.59-6.42 (1H, br m, CH₂NH), 4.50 (1H, ddd, J 11, 6, 1.5, CHNH), 3.30-3.16 (2H, m, CH₂NH), 2.08-2.02 (3H, m, CH₂CO+lactam ring CH), 2.00-1.90 (1H, m, lactam ring CH), 1.86-1.75 (2H, m, lactam ring CH ×2), 1.47-1.31 (2H, br m, lactam ring CH ×2), 1.30-1.17 (16H, m, (CH₂)₈), 0.89 (6H, s, C(CH₃)₂) and 0.84 (3H, t, J 7, CH₂CH₃); δ_{C} (125 MHz, CDCl₃) 175.8, 170.9 (CO), 52.0 (NHCH), 48.4, 42.6, 41.1 (CH₂), 33.3 (CMe₂), 31.9, 31.7, 30.4, 29.7, 29.6, 29.3, 28.9, 27.9 (CH₂), 27.3 (×2) (CH₃), 24.1, 22.6 (CH₂) and 14.1 (CH₃); m/z (M⁺ C₂₀H₃₈N₂O₂ requires 338.2933) 338.2928.

### Example 46

### (E)-Ethyl 2-methyldodec-2-enoate (intermediate)

Decanal (5 mmol) and (carbethoxyethylidene)triphenylphosphorane (10 mmol) were dissolved in CH₂Cl₂ (20 ml) and the reaction was stirred for 18 hours. The solvent was then removed in vacuo and the residue was filter through a plug of silica gel with the aid of 5% diethyl ether in hexanes. The collected eluent was reduced in vacuo to give (E)-ethyl 2-methyldodec-2-enoate as an oil (1.02 g, 88%); νₘₐₓ/cm⁻¹ 1709 (CO), 1651 (C═C); δ_{H} (500 MHz, CDCl₃) 6.73 (1H, tq, J 7.5, 1.5, CH═C), 4.16 (2H, q, J 7, OCH₂), 2.13 (2H, br q, J 7.5, CH₂CH═C), 1.80 (3H, d, J 1.5, CH₃C═CH), 1.45-1.37 (2H, m, chain CH₂), 1.32-1.19 (15H, m, (CH₂)₆+OCH₂CH₃) and 0.85 (3H, t, J 7, (CH₂)₈CH₃); δ_{C} (125 MHz, CDCl₃) 168.3 (CO), 142.4 (CH═C), 127.6 (CH═C), 60.3 (OCH₂), 31.8, 29.5, 29.4 (×2), 29.3, 28.6, 28.5, 22.6 (CH₂), 14.3, 14.1 and 12.3 (CH₃); m/z (MH⁺ C₁₅H₂₉O₂ requires 241.2168) 241.2165.

### Example 47

### (E)-2-Methyldodec-2-enoic acid (Intermediate)

(E)-Ethyl 2-methyldodec-2-enoate (1.43 mmol) was dissolved in ethanol (10 ml), and KOH (10 mmol) in water (5 ml) was added. The reaction was heated at reflux for 18 hours and then cooled. The solvent was removed in vacuo and the residue partitioned between water and hexane. The aqueous layer was acidified with aqueous HCl, and was extracted with diethyl ether. The diethyl ether layer was dried over Na₂SO₄ and reduced in vacuo to give (E)-2-methyldodec-2-enoic acid as a solid (308 mg, >95%); m.p. 28-31° C.; δ_{H} (400 M, CDCl₃) 6.91 (1H, tq, J 7.5, 1.5, CH═C), 2.18 (2H, br q, J 7.5, CH₂CH═C), 1.82 (3H, d, J 1.5, CH₃C═CH), 1.48-1.39 (2H, m, chain CH₂), 1.36-1.19 (12H, m, (CH₂)₆) and 0.88 (3H, t, J 7, (CH₂)₈CH₃) (no OH peak observed).

### Example 48

### (E)-2-Methyldodec-2-enoyl chloride (Intermediate)

(E)-2-Methyldodec-2-enoic acid (1.43 mmol) was dissolved in CH₂Cl₂ (20 ml) oxalyl chloride (1 ml) and dimethyl formamide (1 drop) was added. After 1 hour the reaction was reduced in vacuo to give crude (E)-2-methyldodec-2-enoyl chloride which was used directly in the synthesis of (S)-(E)-3-(2′-methyldodec-2′-enoyl)amino-caprolactam.

### Example 49

### (S)-(E)-3-(2′-Methyldodec-2′-enoyl)amino-caprolactam

(S,S)-3-amino-caprolactam hydro-pyrrolidine-5-carboxylate 2 (2 mmol) and Na₂CO₃ (6 mmol) in water (15 ml) were added to a solution of (E)-2-methyldodec-2-enoyl chloride (1.43 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from hexane to give (S)-(E)-3-(2′-methyldodec-2′-enoyl)amino-caprolactam (297 mg, 65%); m.p. (hexanes) 99-100° C.; νₘₐₓ/cm⁻¹ 3282 (NH), 1656, 1622 (CO and C═C), 1497 (NH); [α]_{D}²⁵ (c=1, CHCl₃) +38.2; δ_{H} (500 M, CDCl₃) 7.15 (1H, d, J 5.5, NHCH), 6.48-6.35 (2H, m, NHCH₂+CH═C), 4.54 (1H, ddd, J 11, 5.5, 1.5, NHCH), 3.33-3.17 (2H, m, CH₂NH), 2.14-2.05 (3H, m, CH₂CH═C+lactam ring CH), 2.02-1.93 (1H, m, lactam ring CH), 1.88-1.77 (5H, m, lactam ring CH ×2+CH₃C═CH), 1.47-1.31 (4H, br m, lactam ring CH ×2+chain CH₂), 1.31-1.17 (12H, m, (CH₂)₆) and 0.85 (3H, t, J 7, CH₂CH₃); δ_{C} (125 MHz, CDCl₃) 175.9, 168.2 (CO), 136.9 (CH═C), 130.2 (CH═C), 52.3 (NHCH), 42.2 (NHCH₂), 31.8, 31.6, 29.5, 29.4 (×2), 29.3, 28.9, 28.7, 28.3, 27.9, 22.6 (CH₂), 14.1 and 12.4 (CH₃).

### Example 50(a)

### (3S,2′R) and

### Example 50(b)

### (3S,2′S)-3-(2′-Methyldodecanoyl)amino-caprolactam

(S)-(E)-3-(2′-Methyldodec-2′-enoyl)amino-caprolactam (0.5 mmol) and Pd(OH)₂ (20% on carbon) were added to methanol (10 ml) and the mixture was stirred for 18 hours at ambient temperature under an atmosphere of hydrogen. The reaction was then filtered, and the solvent removed in vacuo to give (3S,2′R) and (3S,2′S)-3-(2′-methyldodecanoyl)amino-caprolactam as a solid (160 mg, >95%); νₘₐₓ/cm⁻¹ 3313 (NH), 1671, 1636 (CO), 1515 (NH); δ_{H} (500 MHz, CDCl₃) 6.91 (2H, d, J 5.5, CHNH, both isomers), 6.55 (2H, br s, CH₂NH, both isomers), 4.57-4.47 (2H, m, CHNH, both isomers), 3.34-3.18 (4H, m, CH₂NH, both isomers), 2.29-2.14 (2H, CH₃CHCO, both isomers), 2.07 (2H, br d, J 13.5, lactam ring CH, both isomers), 2.02-1.94 (2H, m, lactam ring CH, both isomers), 1.89-1.76 (4H, m, lactam ring CH ×2, both isomers), 1.67-1.57 (2H, m, chain CH, both isomers), 1.51-1.33 (6H, m, lactam ring CH ×2+side chain CH₂, both isomers), 1.32-1.18 (32H, m, (CH₂)₈, both isomers), 1.13 (3H, d, J 7, CHCH₃, one isomer), 1.11 (3H, d, J 7, CHCH₃, one isomer) and 0.87 (6H, t, J 7.5, CH₃, both isomers); δ_{C} (125 MHz, CDCl₃) 175.9 (×2), 175.8 (×2) (CO, both isomers), 52.0, 51.9 (NCH), 42.1 (×2) (NCH₂, both isomers), 41.3, 41.2 (CHCH₃), 34.5, 34.1, 31.9 (×2), 31.8, 31.7, 29.6 (×6), 29.5 (×2), 29.3 (×2), 28.9 (×2), 28.0, 27.9, 27.4 (×2), 22.6 (×2) (CH₂) 17.8, 17.6 and 14.1 (×2) (CH₃); m/z (MH⁺ C₁₉H₃₇N₂O₂ requires 325.2855) 325.2858.

### Example 51

### (4S,2′S,3′R)-4-Benzyl-3-(3′-hydroxy-2′-methyldecanoyl)-oxazolidin-2-one (Intermediate)

This aldol reaction was performed according to published method (Crimmins, M. T; She, J.; Synlett, 2004, 1371-1374). (S)-4-Benzyl-3-propionyl-oxazolidin-2-one (5 mmol) (synthesised according to the method of Evans et al. Tetrahedron Lett., 1987, 28, 1123) was dissolved in CH₂Cl₂ (25 ml) and the solution was cooled to −20° C. under an atmosphere of dry nitrogen and TiCl₄ (5.25 mmol) was added. After 15 minutes, diisopropylethylamine (5.5 mmol) was added. After a further 40 minutes N-methyl-pyrrolidin-2-one (5.25 mmol) was added. After a further 10 minutes, decanal (5.5 mmol) was added and the reaction was stirred for 1 hour. Ammonium chloride solution was added and the reaction mixture was washed with pH 2 buffer (0.5 M Na₂SO₄/0.5 M NaHSO₄). The organic layer was dried over Na₂SO₄ and reduced in vacuo. The crude product was chromatographed on silica gel (10% to 33% ethyl acetate in hexane) to give (4S,2′S,3′R)-4-benzyl-3-(3′-hydroxy-2′-methyldecanoyl)-oxazolidin-2-one as an oil (1.34 g, 69%); νₘₐₓ/cm⁻¹ 1778 (NCO₂), 1697 (CON); δ_{H} (500 MHz, CDCl₃) 7.35-7.30 (2H, m, meta-Ph), 7.29-7.24 (1H, m, para-Ph), 7.21-7.17 (2H, m, ortho-Ph), 4.69 (1H, ddt, J 9.5, 7.5, 3.5, CHN), 4.21 (1H, t, J 9, OCHH), 4.17 (1H, dd, J 9, 3, OCHH), 3.93 (1H, ddd, J 7, 4.5, 3, CHOH), 3.75 (1H, qd, J 7, 2.5, CHCH₃), 3.24 (1H, dd, J 13.5, 3.5, CHHPh), 2.87 (1H, br s, CHOH), 2.78 (1H, dd, J 13.5, 9.5, CHHPh), 1.56-1.20 (19H, m, (CH₂)₈+CHCH₃) and 0.86 (3H, t, J 7, CH₂CH₃); δ_{C} (125 MHz, CDCl₃) 177.6 (CCO), 153.0 (OCO), 135.0 (ipso-Ph), 129.4, 129.0 (ortho-+meta-Ph), 127.4 (para-Ph), 71.5 (CHOH), 66.1 (OCH₂), 55.1 (NCH), 42.1 (CHCH₃), 37.8, 33.8, 31.9, 29.6 (×3), 29.3, 26.0, 22.7 (CH₂), 14.1 and 10.3 (CH₃); m/z (MH⁺ C₂₃H₃₆NO₄ requires 390.2644) 390.2641.

### Example 52

### (4R,2′R,3′S)-4-Benzyl-3-(3′-hydroxy-2′-methyldecanoyl)-oxazolidin-2-one (Intermediate)

(R)-4-Benzyl-3-propionyl-oxazolidin-2-one was converted into (4R,2′R,3′S)-4-benzyl-3-(3′-hydroxy-2′-methyldecanoyl)-oxazolidin-2-one according to the above procedure. NMR spectroscopic data is identical; m/z (MH⁺ C₂₃H₃₆NO₄ requires 390.2644) 390.2638.

### Example 53

### (2S,3R)-3-Hydroxy-2-methyldecanoic acid (Intermediate)

(4S,2′S,3′R)-4-Benzyl-3-(3′-hydroxy-2′-methyldecanoyl)-oxazolidin-2-one (1.42 mmol) was dissolved in THF (10 ml). Water (2 ml), aqueous hydrogen peroxide (8M, 0.5 mmol) and LiOH.H₂O (3 mmol) were added, and the reaction was stirred for 18 hours. Na₂SO₃ (10 mmol) was added and the reaction was extracted with ethyl acetate. The aqueous layer was then acidified with pH 2 buffer (0.5 M Na₂SO₄/0.5 M NaHSO₄), and extracted with diethyl ether. The diethyl ether layer was dried over Na₂SO₄ and reduced in vacuo to give crude (2S,3R)-3-hydroxy-2-methyldecanoic acid; δ_{H} (400 z, CDCl₃) 3.96-3.89 (1H, m, CHOH), 2.59 (1H, dq, J 7, 3, CHCH₃), 1.54-1.36 (2H, m, CH₂), 1.36-1.22 (14H, m, (CH₂)₇) and 1.20 (3H, d, J 7, CHCH₃). This material was used directly in the synthesis of (3S,2′S,3′R)-3-(3′-hydroxy-2′-methyldecanoyl)amino-caprolactam.

### Example 54

### (2R,3S)-3-Hydroxy-2-methyldecanoic acid (Intermediate)

(2R,3S)-3-Hydroxy-2-methyldecanoic acid was prepared from (4R,2′R,3′S)-4-benzyl-3-(3′-hydroxy-2′-methyldecanoyl)-oxazolidin-2-one according to the above procedure.

### Example 55

### (3S,2′S,3′R)-3-(3′-Hydroxy-2′-methyldecanoyl)amino-caprolactam

(2S,3R)-3-Hydroxy-2-methyldecanoic acid (1.40 mmol) was dissolved in MeOH (10 ml), and (S)-3-amino-caprolactam hydrochloride (1.50 mmol) and triethylamine (2 mmol) were added. The reaction was cooled to 0° C. and 4-(4,6-dimethoxy[1,3,5]triazin-2-yl)-4-methyl-morpholinium chloride (1.40 mmol) was added. The reaction was stirred for 4 hours, and then the solvent was removed in vacuo. The residue was partitioned between ethyl acetate and water. The ethyl acetate layer was washed with dilute aqueous HCl and dilute aqueous NaOH, and then dried over Na₂SO₄. The solvent was removed in vacuo and the residue was recrystallised form ethyl acetate/hexane to give (3S,2′S,3′R)-3-(3′-hydroxy-2′-methyldecanoyl)amino-caprolactam as a solid (341 mg, 72%) m.p. (hexanes) 88-91° C.; νₘₐₓ/cm⁻¹ 3313 (NH), 1628 (CO), 1480 (NH); [α]_{D}²⁵ (c=0.5, CHCl₃) +40.8; δ_{H} (500 MHz, CDCl₃) 7.08 (1H, d J 5.5, NHCH), 6.51 (1H, br s, NHCH₂), 4.57 (1H, ddd, J 11, 6.5, 1, NCH), 3.83 (1H, dt, J 8, 4, CHOH), 3.36-3.21 (2H, m, NCH₂), 2.40 (1H, dq, J 7, 3, CHCH₃), 2.12-1.96 (2H, m, lactam CH ×2), 1.90-1.76 (2H, m, lactam CH ×2), 1.55-1.34 (4H, m, lactam CH ×2+chain CH₂), 1.34-1.21 (14H, m, (CH₂)₇), 1.17 (3H, d, J 7, CHCH₃) and 0.88 (3H, t, J 7, CH₂CH₃) (OH not observed); δ_{C} (125 MHz, CDCl₃) 175.8, 175.7 (CO), 72.1 (CHOH), 52.0 (NCH), 44.6 (CHCH₃), 42.1 (NCH₂), 33.4, 31.9, 31.3, 29.6 (×2), 29.5, 29.3, 28.8, 27.9, 26.1, 22.7 (CH₂), 14.1 and 11.2 (CH₃); m/z (MH⁺ C₁₉H₃₇N₂O₃ requires 341.2804) 341.2776.

### Example 56

### (3S,2′R,3′S)-3(3′-Hydroxy-2′-methyldecanoyl)amino-caprolactam

(2R,3S)-3-Hydroxy-2-methyldecanoic acid (1.40 mmol), (S)-3-amino-caprolactam hydrochloride (1.50 mmol), triethylamine (2 mmol), and 4-(4,6-dimethoxy[1,3,5]triazin-2-yl)-4-methyl-morpholinium chloride (1.40 mmol) were reacted together, as above to produce (3S,2′R,3′S)-3-(3′-hydroxy-2′-methyldecanoyl)amino-caprolactam which was recrystallised from ethyl acetate/hexane (86 mg, 18%); m.p. (hexanes) 118-121° C.; νₘₐₓ/cm⁻¹ 3294 (NH), 1667, 1613 (CO), 1533 (NH); [α]_{D}²⁵ (c=0.5, CHCl₃) +14.8; δ_{H} (500 MHz, CDCl₃) 7.11 (1H, d, J 6, NHCH), 6.54 (1H, br s, NHCH₂), 4.53 (1H, ddd, J 11, 6.5, 1.5, NCH), 3.87-3.80 (1H, m, CHOH), 3.70 (1H, br s, OH), 3.34-3.20 (2H, m, NCH₂), 2.37 (1H, dq, J 7, 3, CHCH₃), 2.11-1.96 (2H, m, lactam CH ×2), 1.90-1.76 (2H, m, lactam CH ×2), 1.55-1.21 (18H, m, lactam CH ×2+chain (CH₂)₈), 1.16 (3H, d, J 7, CHCH₃) and 0.88 (3H, t, J 7, CH₂CH₃); δ_{C} (125 MHz, CDCl₃) 175.9, 175.7 (CO), 72.0 (CHOH), 52.1 (NCH), 44.8 (CHCH₃), 42.1 (NCH₂), 33.7, 31.9, 31.4, 29.6 (×2), 29.5, 29.3, 28.8, 27.9, 26.0, 22.7 (CH₂), 14.1 and 10.7 (CH₃); m/z (MH⁺ C₁₉H₃₇N₂O₃ requires 341.2804) 341.2803.

### Example 57

### Methyl 2,2-dimethyl-3-hydroxy decanoate (Intermediate)

Butyllithium (2.5 M in hexanes, 50 mmol) was added to a solution of diisopropylamine (50 mmol) in dry THF (200 ml) at −78° C. under an atmosphere of dry nitrogen. The reaction was stirred for 30 minutes, and then methyl isobutyrate (50 mmol) was added. After 45 minutes, decanal (50 mmol) was added and the reaction was allowed to warm to ambient temperature over 18 hours. After the addition of saturated aqueous ammonium chloride (10 ml), the reaction solvent was removed in vacuo and the residue was partitioned between hexanes and pH 2 buffer (0.5 M Na₂SO₄/0.5 M NaHSO₄). The organic layer was dried over Na₂SO₄ and the solvent was removed to give methyl 2,2-dimethyl-3-hydroxy decanoate as an oil (9.98 g, 77%); δ_{H} (400 MHz, CDCl₃) 3.70 (3H, s, OCH₃), 3.69 (1H, dd, J 10, 2, CHOH), 1.68-1.20 (16H, m, (CH₂)₈), 1.19 (3H, s, CCH₃), 1.17 (3H, s, CCH₃) and 0.88 (3H, t, J 7, CH₂CH₃) (no OH observed).

### Example 58

### 2,2-Dimethyl-3-hydroxy decanoic acid (Intermediate)

Methyl 2,2-dimethyl-3-hydroxy decanoate (20 mmol) was dissolved in EtOH (80 ml) and a solution of KOH (40 mmol) in water (20 ml) was added. The reaction was heated at reflux for 18 hours, and then the reaction was allowed to cool. The solvent was removed in vacuo and the residue was partitioned between water and diethyl ether. The aqueous layer was then acidified with pH 2 buffer (0.5 M Na₂SO₄/0.5 M NaHSO₄) and extracted with diethyl ether. The solution was dried over Na₂SO₄ and reduced in vacuo to give 2,2-dimethyl-3-hydroxy decanoic acid which solidified on standing; m.p. 39-41° C.; δ_{H} (400 MHz, CDCl₃) 3.64 (1H, dd, J 10, 2, CHOH), 1.67-1.12 (22H, m, (CH₂)₈+C(CH₃)₂) and 0.88 (3H, t, J 7, CH₂CH₃).

### Example 59(a)

### (3S,3′R) and

### Example 59(b)

### (3S,3′S)-3-(3′-Hydroxy-2′,2′-dimethyldecanoyl)amino-caprolactam

2,2-Dimethyl-3-hydroxy decanoic acid (1.77 mmol) and 1-hydroxybenzotriazole monohydrate (1.77 mmol) were dissolved in THF (10 ml). 1-[3-(Dimethylamino)propyl]-3-ethylcarbodiimide hydrochloride (1.77 mmol) was added and the reaction was stirred at ambient temperature for 4 hours. A solution of (S,S)-3-amino-caprolactam hydro-pyrrolidine-5-carboxylate 2 (2 mmol) and Na₂CO₃ (6 mmol) in water (15 ml) was added and the reaction was stirred for 18 hours. The reaction solvent was then removed in vacuo and the residue was partitioned between water and ethyl acetate. The ethyl acetate layer was washed with pH 2 buffer (0.5 M Na₂SO₄/0.5 M NaHSO₄) and dilute aqueous sodium hydroxide, and then dried over Na₂SO₄ and reduced in vacuo. The residue was chromatographed on silica gel (25% ethyl acetate in hexanes to 100% ethyl acetate) to give a mixture of (3S,3′R) and (3S,3′S)-3-(3′-hydroxy-2′,2′-dimethyldecanoyl)amino-caprolactams (557 mg, 88%); δ_{H} (500 MHz, CDCl₃) 7.28 (1H, d, J 6, NHCH one isomer), 7.25 (1H, d, J 6, NHCH, one isomer), 6.62-6.48 (1H, br m, NHCH₂, both isomers), 4.53-4.42 (1H, m, NCH, both isomers), 3.77 (1H, br d, J, 6, OH, one isomer), 3.63 (1H, br d, J, 6, OH, one isomer), 3.47-3.36 (1H, m, CHOH, both isomers), 3.32-3.17 (2H, m, NCH₂, both isomers), 2.07-1.92 (2H, m, lactam CH ×2, both isomers), 1.87-1.71 (2H, m, lactam CH ×2, both isomers), 1.60-1.17 (21H, m, lactam CH ×2+chain (CH₂)₈+CH₃, both isomers), 1.14 (3H, s, CCH₃, both isomers) and 0.84 (3H, t, J 7, CH₂CH₃, both isomers); δ_{C} (125 MHz, CDCl₃) 177.6, 177.2, 175.8 (CO, both isomers), 77.8, 77.4 (CHOH), 52.1 (NCH, both isomers), 45.9, 45.8 (C(CH₃)₂), 42.1, 42.0 (NCH₂), 31.9 (×2) 31.6, 31.3, 30.9, 29.6 (×4), 29.3, 28.8, 27.9, 26.7, 26.6, 22.6 (CH₂), 23.7, 23.5, 21.1, 20.4 and 14.1 (CH₃);

### Example 60

### 2,2-Dimethyl-3-(tetrahydropyran-2-yloxy)-propionic acid (Intermediate)

2,2-Dimethyl-3-hydroxy propionic acid (100 mmol) and 3,4-dihydro-2H-pyran (210 mmol) were dissolved in dichloromethane (50 ml), and para-toluenesulfonic acid (10 mg) was added and the reaction was stirred at ambient temperature for 3 hours. The reaction solvent was then removed and the residue was dissolved in ethanol (100 ml). A solution of KOH (120 mmol) in water (30 ml) was added and the reaction was heated at reflux for 18 hours. The reaction solvent was removed in vacuo and the residue was partitioned between water and diethyl ether. The aqueous layer was acidified with pH 2 buffer (0.5 M Na₂SO₄/0.5 M NaHSO₄) and then extracted with diethyl ether. The diethyl ether layer was then dried over Na₂SO₄ and the solvent was removed in vacuo to give 2,2-dimethyl-3-(tetrahydropyran-2-yloxy)-propionic acid as an oil (20.0 g, >95%); δ_{H} (400 M, CDCl₃) 4.62 (1H, t, J 3.5, CHO), 3.82 (1H, ddd, J 12, 9, 3, ring CH₂O), 3.75 (1H, d, J 12, chain CH₂O), 3.55-3.46 (1H, m, ring CH₂O), 3.40 (1H, d, J 12, chain CH₂O), 1.90-1.45 (6H, m, (CH₂)₃), 1.25 (3H, s, CH₃) and 1.23 (3H, s, CH₃).

### Example 61

### (S)-(2′,2′-Dimethyl-3′-hydroxy-propionyl)amino-caprolactam

2,2-Dimethyl-3-(tetrahydropyran-2-yloxy)-propionic acid (4.65 mmol), 1-hydroxybenzotriazole monohydrate (4.65 mmol) and carbonyl diimidazole (4.50 mmol) were dissolved in THF (30 ml) and the reaction was heated at reflux for 4 hours. After the reaction was cooled to ambient temperature, a solution of (S,S)-3-amino-caprolactam hydro-pyrrolidine-5-carboxylate 2 (5 mmol) and Na₂CO₃ (15 mmol) in water (30 ml) was added and the reaction was stirred for 18 hours. The THF was then removed from the reaction by distillation in vacuo and the aqueous layer was extracted with ethyl acetate. The ethyl acetate layer was dried over Na₂SO₄ and reduced in vacuo. The residue was dissolved in MeOH, and acetyl chloride (1 ml) was added. The reaction was stirred at ambient temperature for 18 hours, and then reduced in vacuo to give (S)-(2′-dimethyl-3′-hydroxy propionyl)amino-caprolactam as a solid (854 mg, 83%); m.p. 97-99° C.; [α]_{D}²⁵ (c=0.5, CHCl₃) +30.0; δ_{H} (400 M, CDCl₃) 7.24 (1H, d, J 5.0, CHNH), 6.38 (11H, br s, CH₂NH), 4.49 (1H, dd, J 10, 6, CHNH), 3.54 (1H, d, J 11, CHHOH), 3.49 (1H, d, J 11, CHHOH), 3.33-3.20 (2H, m, CH₂NH), 2.03-1.96 (2H, m, 2× ring CH), 1.87-1.72 (2H, m, 2× ring CH), 1.50-1.30 (2H, m, 2× ring CH), 1.20 (3H, s, CH₃) and 1.18 (3H, s, CH₃); δ_{C} (125 MHz, CDCl₃) 177.2, 176.0 (CO), 69.9 (CHOH), 52.1 (NHCHCO), 43.2 (CCO), 41.9 (CH₂N), 31.1, 28.8, 27.9 (CH₂ lactam), 22.4 and 22.3 (CH₃);

### Example 62

### (S)-(3′-Chloro-2′-(chloromethyl)-2′-methylpropionyl)amino-caprolactam

(S,S)-3-amino-caprolactam hydro-pyrrolidine-5-carboxylate 2 (5 mmol) and Na₂CO₃ (15 mmol) in water (15 ml) were added to a solution of 3,3′-dichloropivaloyl chloride (5 mmol) in dichloromethane (15 ml) at ambient temperature and the reaction was stirred for 12 hours. The organic layer was then separated and the aqueous phase was extracted with additional dichloromethane (2×25 ml). The combined organic layers were dried over Na₂SO₄ and reduced in vacuo. The residue was recrystallised from hexane to give (S)-(3′-chloro-2′-(chloromethyl)-2′-methylpropionyl)amino-caprolactam (973 mg, 69%); m.p. (hexanes) 95-96° C.; [α]_{D}²⁵ (c=0.5, CHCl₃) +16.4; δ_{H} (500 MHz, CDCl₃) 7.33 (1H, d, J 5.0, CHNH), 6.82-6.62 (1H, br m, CH₂NH), 4.49 (1H, ddd, J 11, 5.5, 1.5, CHNH), 3.78 (1H, d, J 11, CHHCl), 3.74 (1H, d, J 11, CHHCl), 3.69 (1H, d, J 11, CHHCl), 3.66 (1H, d, J 11, CHHCl), 3.29-3.17 (2H, m, CH₂NH), 2.05 (1H, br s, J 13.5, ring CH), 2.01-1.93 (1H, m, ring CH), 1.87-1.71 (2H, m, 2× ring CH) and 1.49-1.31 (5H, m, 2× ring CH+CH₃); δ_{C} (125 MHz, CDCl₃) 175.4, 170.6 (CO), 52.6 (NHCHCO), 49.1 (CCO), 48.7, 48.6 (CH₂Cl), 42.1 (CH₂N), 31.1, 28.8, 27.9 (CH₂ lactam) and 18.9 (CH₃).

### Pharmacological Study of the Products of the Invention

### Inhibition of MCP-1 Induced Leukocyte Migration

### Assay Principle

The biological activity of the compounds of the current invention may be demonstrated using any of abroad range of functional assays of leukocyte migration in vitro, including but not limited to Boyden chamber and related transwell migration assays, under-agarose migration assays and direct visualisation chambers such as the Dunn Chamber.

For example, to demonstrate the inhibition of leukocyte migration in response to chemokines (but not other chemoattractants) the 96-well format micro transwell assay system from Neuroprobe (Gaithersburg, Md., USA) has been used. In principle, this assay consists of two chambers separated by a porous membrane. The chemoattractant is placed in the lower compartment and the cells are placed in the upper compartment. After incubation for a period at 37° C. the cells move towards the chemoattractant, and the number of cells in the lower compartment is proportional to the chemoattractant activity (relative to a series of controls).

This assay can be used with a range of different leukocyte populations. For example, freshly prepared human peripheral blood leukocytes may used. Alternatively, leukocyte subsets may be prepared, including polymorphonuclear cells or lymphocytes or monocytes using methods well known to those skilled in the art such as density gradient centrifugation or magnetic bead separations.

Alternatively, immortal cell lines which have been extensively validated as models of human peripheral blood leukocytes may be used, including, but not limited to THP-1 cells as a model of monocytes or Jurkat cells as model of naïve T cells.

Although a range of conditions for the assay are acceptable to demonstrate the inhibition of chemokine-induced leukocyte migration, a specific example is hereby provided.

### Materials

The transwell migration systems are manufactured by Neuroprobe, Gaithersburg, Md., USA.

The plates used are ChemoTx plates (Neuroprobe 101-8) and 30 μl clear plates (Neuroprobe MP30).

Geys' Balanced Salt Solution is purchased from Sigma (Sigma G-9779).

Fatty acid-free BSA is purchased from Sigma (Sigma A-8806).

MTT, i.e. 3-(4,5-dimethylthiazol-2-yl)-2,5-diphenyltetrazolium bromide, is purchased from Sigma (Sigma M-5655).

RPMI-1640 without phenol red is purchased from Sigma (Sigma R-8755).

The THP-1 cell line (European Cell culture Collection) were used as the leukocyte cell population.

### Test Protocol

The following procedure is used for testing the invention compounds for MCP-1 induced leukocyte migration:

First, the cell suspension to be placed in the upper compartment is prepared. The THP-1 cells are pelleted by centrifugation (770×g; 4 mins) and washed with Geys Balanced Salt Solution with 1 mg/ml BSA (GBSS+BSA). This wash is then repeated, and the cells repelleted before being resuspended in a small volume of GBSS+BSA for counting, for example using a standard haemocytometer.

The volume of GBSS+BSA is then adjusted depending on the number of cells present so that the cells are at final density of 4.45×10⁶ cells per ml of GBSS+BSA. This ensures that there are 100,000 THP-1 cells in each 25 μl of the solution that will be placed in the upper chamber of the plate.

To test a single compound for its ability to inhibit MCP-1 induced migration, it is necessary to prepare two lots of cells. The suspension of THP-1 cells at 4.45×10⁶ cells/ml is divided into two pots. To one pot the inhibitor under test is added at an appropriate final concentration, in an appropriate vehicle (for example at 1 μM in not more than 1% DMSO). To the second pot an equal volume of GBSS+BSA plus vehicle as appropriate (e.g. not more than 1% DMSO) is added to act as a control.

Next, the chemoattractant solution to be placed in the lower compartment is prepared. MCP-1 is diluted in GBSS+BSA to give a final concentration of 25 ng/ml. This is divided into two pots, as for the cell suspension. To one pot, the test compound is added to the same final concentration as was added to the cell suspension, while to the other pot an equal volume of GBSS+BSA plus vehicle as appropriate (e.g. not more than 1% DMSO) is added.

Note that the volume of liquid that needs to be added to make the addition of the text compound needs to be taken into account, when establishing the final concentration of MCP-1 in the solution for the lower compartment and the final concentration of cells in the upper compartment.

Once the chemoattractant solutions for the lower wells and cell solutions for the upper chambers have been prepared, the migration chamber should be assembled. Place 29 μl of the appropriate chemoattractant solution into the lower well of the chamber. Assays should be performed with at least triplicate determinations of each condition. Once all the lower chambers have been filled, apply the porous membrane to the chamber in accordance with the manufacturer's instructions. Finally, apply 25 μl of the appropriate cell solution to each upper chamber. A plastic lid is placed over the entire apparatus to prevent evaporation.

The assembled chamber is incubated at 37° C., 5% CO₂, for 2 hours. A suspension of cells in GBSS+BSA is also incubated under identical conditions in a tube: these cells will be used to construct a standard curve for determining the number of cells that have migrated to the lower chamber under each condition.

At the end of the incubation, the liquid cell suspension is gently removed from the upper chamber, and 20 μl of ice-cold 20 mM EDTA in PBS is added to the upper chamber, and the apparatus is incubated at 4° C. for 15 mins. This procedure causes any cells adhering to the underside of the membrane to fall into the lower chamber.

After this incubation the filter is carefully flushed with GBSS+BSA to wash off the EDTA, and then the filter is removed.

The number of cells migrated into the lower chamber under each condition can then be determined by a number of methods, including direct counting, labelling with fluorescent or radioactive markers or through the use of a vital dye. Typically, we utilise the vital dye MTT. 3 μl of stock MTT solution are added to each well, and then the plate is incubated at 37° C. for 1-2 hours during which time dehydrogenase enzymes within the cells convert the soluble MTT to an insoluble blue formazan product that can be quantified spectrophotometrically.

In parallel, an 8-point standard curve is set up. Starting with the number of cells added to each upper chamber (100,000) and going down in 2-fold serial dilutions in GBSS+BSA, the cells are added to a plate in 25 μl, with 3 μl of MTT stock solution added.

The standard curve plate is incubated along side the migration plate.

At the end of this incubation, the liquid is carefully removed from the lower chambers, taking care not to disturb the precipitated formazan product. After allowing to air dry briefly, 20 μl of DMSO is added to each lower chamber to solubilise the blue dye, and absorbance at 595 nm is determined using a 96-well plate reader. The absorbance of each well is then interpolated to the standard curve to estimate the number of cells in each lower chamber.

The MCP-1 stimulated migration is determined by subtracting the average number of cells that reached the lower compartment in wells where no MCP-1 was added from the average number of cells that reached the lower compartment where MCP-1 was present at 25 ng/ml.

The impact of the test substance is calculated by comparing the MCP-1-induced migration which occurred in the presence or absence of various concentrations of the test substance. Typically, the inhibition of migration is expressed as a percentage of the total MCP-1 induced migration which was blocked by the presence of the compound. For most compounds, a dose-response graph is constructed by determining the inhibition of MCP-1 induced migration which occurs at a range of different compound concentrations (typically ranging from 1 nM to 1 μM or higher in the case of poorly active compounds). The inhibitory activity of each compound is then expressed as the concentration of compound required to reduce the MCP-1-induced migration by 50% (the ED₅₀ concentration).

### Results

The compounds of examples 1 to 7 and 9 to 34 and 39, 41, 42, 45, 49, 50, 55, 56, 61 and 62 were tested and were shown to have an ED₅₀ of 100 nM or less in this test.

### Enantioselectivity

The (S)- and (R)-enantiomers of three different members of the aminocaprolactam series were synthesised to determine whether the biological activity showed enantioselectivity.

The comparison was made between the compounds of examples 1 and 7, between the compounds of examples 10 and 11, and between the compounds of examples 12 and 17.

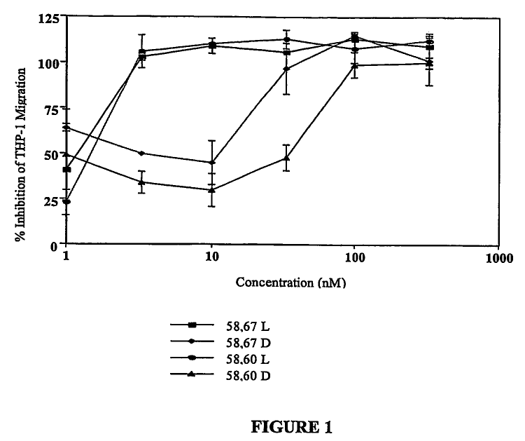

The dose-response curves for each of the four compounds of examples 1, 7, 10 and 11 as inhibitors of MCP-1 induced THP-1 cell migration were determined using the transwell migration assay and are shown in FIG. 1. In both cases, the (S)-enantiomer was significantly (10-50 fold) more active than the (R)-enantiomer.

Very similar data was obtained using the compounds of examples 12 and 17, such that the (S)-enantiomer was significantly (10-50 fold) more active than the (R)-enantiomer.

These data, for three example members of the aminocaprolactam series, demonstrate that for the application of the compounds of the present invention as anti-inflammatory agents in vivo it is preferable to use the pure (S)-enantiomer of the compound, rather than the racemic mixture of the two enantiomers or the pure (R)-enantiomer.

### In Vivo Activity of Compounds of the Invention:

The anti-inflammatory activity of the compounds of the invention was determined in vivo using a sub-lethal LPS-induced endotoxemia model. Adult male CD-1 mice (n=6 per group) were pretreated with various agents (vehicle, compounds of the invention or positive control agents such as the steroid dexamethasone) by sub-cutaneous injection 30 minutes prior to an acute inflammatory challenge with 750 μg of bacterial lipopolysaccharide (from E. Coli 0111:B4; Sigma catalog #L-4130) via the intraperitoneal route. The vehicle in each case was 0.6% DMSO, 1% carboxymethyl cellulose, or alternatively 1% carboxymethylcellulose alone. For some of the compounds, this formulation results in a finely divided suspension or slurry rather than a clear solution. Two hours after LPS challenge, the animals were sacrificed and blood was drawn by cardiac puncture. The level of the pro-inflammatory cytokine TNF-alpha was determined using the Quantikine M ELISA (R&D Systems) for murine TNF-alpha, and reported as the mean ±standard error for each group.

Mice which did not receive the LPS challenge had very low circulating levels of TNF-alpha (typically 10 pg/ml). By 2 hours after the LPS challenge, this had increased by more than 1,000 fold to an average of 20,000 pg/ml, representing a sensitive index of inflammatory activation. Pre-treatment with known anti-inflammatory drugs (such as the steroid dexamethasone) reduced the stimulation of TNF-alpha by up to 85-95%, depending on the dose given.

Compounds 7, 9, 10, 12 and 20 were all tested in this model. All five compounds were able to block TNF-alpha stimulation to a similar extent to dexamethasone, when given at a suitable dose. All five compounds were maximally active at a dose below 1 mg/kg.

In a separate series of experiments, the compounds of the invention were administered to animals as an oral suspension, formulated in the same way as for the sub-cutaneous dosing experiments, followed 1 hour later with the LPS challenge exactly as described above. Compounds 7, 9, 10, 12 and 20 were all tested in this model, and all five compounds were able to block TNF-alpha stimulation when administered via the oral route at a suitable dose. All five compounds were maximally active at a dose below 30 mg/kg.

1. Use of a compound of general formula (I) or a pharmaceutically acceptable salt thereof, for the preparation of a medicament intended to treat an inflammatory disorder: 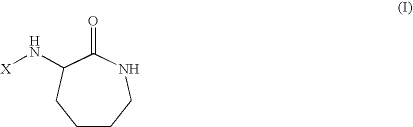
wherein
X is —CO—R¹ or —SO₂—R²,
R¹ is an alkyl, haloalkyl, alkoxy, haloalkoxy, alkenyl, alkynyl or alkylamino radical of 4 to 20 carbon atoms (for example of 5 to 20 carbon atoms, of 8 to 2b carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms); and
R² is an alkyl radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, and of 13 to 17 carbon atoms); or
alternatively R¹ and R² are selected independently from a peptido radical having from 1 to 4 peptidic moieties linked together by peptide bonds.

2. Use of a compound of formula (I′) or a pharmaceutically acceptable salt thereof, for the preparation of a medicament intended to treat an inflammatory disorder: 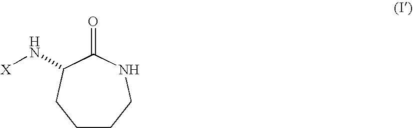
wherein X has the same meaning as above.

3. A pharmaceutical composition comprising, as active ingredient, a compound of formula (I) or a pharmaceutically acceptable salt thereof, and at least one pharmaceutically acceptable excipient and/or carrier: 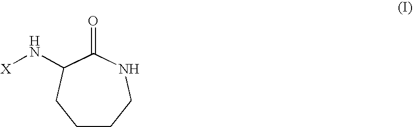
wherein
X is —CO—R¹ or —SO₂—R²,
R¹ is an alkyl, haloalkyl, alkoxy, haloalkoxy, alkenyl, alkynyl or alkylamino radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms); and
R² is an alkyl radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, and of 13 to 17 carbon atoms); or
alternatively R¹ and R² may be selected independently from a peptido radical having from 1 to 4 peptidic moieties linked together by peptide bonds (for example a peptido radical of 1 to 4 amino acid residues).

4. A pharmaceutically acceptable composition comprising active ingredient, a compound of formula (I′) or a pharmaceutically acceptable salt thereof, and at least one pharmaceutically acceptable excipient and/or carrier:

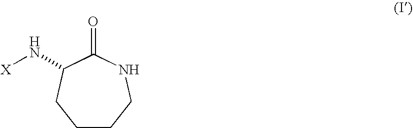

5. A compound of general formula (I): 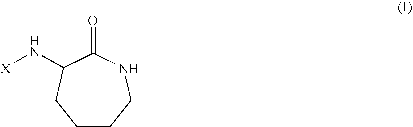
wherein
X is —CO—R¹ or —SO₂—R²,
R¹ is an alkyl, haloalkyl, alkoxy, haloalkoxy, alkenyl, alkynyl or alkylamino radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms); and
R² is an alkyl radical of 4 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, and of 13 to 17 carbon atoms); or
alternatively R¹ and R² are selected independently from a peptido radical having from 1 to 4 peptidic moieties linked together by peptide bonds.

6. The compound of general formula (I′): 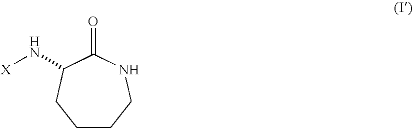
wherein X has the same meaning in claim 5.

7. The compounds, compositions and uses of the compounds of general formula (I) or (I′), or their pharmaceutically acceptable salts, according to claim 1, wherein the alkyl, haloalkyl, alkoxy, haloalkoxy, alkenyl, alkynyl or alkylamino part of the R¹ radical is linear.

8. The compounds, compositions and uses of the compounds of general formula (I) or (I′), or their pharmaceutically acceptable salts, according to claim 1, wherein the alkyl, haloalkyl, alkoxy, haloalkoxy, alkenyl, alkynyl or alkylamino part of the R¹ radical is branched.

9. The compounds, compositions and uses of the compounds of general formula (I) or (I′), or their pharmaceutically acceptable salts, according to claim 1, wherein the alkyl, haloalkyl, alkoxy, haloalkoxy, alkenyl, alkynyl or alkylamino part of the R¹ radical is either linear or is branched but contains a linear chain of at least 8 or at least 10 carbon atoms.

10. The compounds, compositions and uses according to claim 8 wherein the R1 radical has an alpha-carbon (2-position in X) which is substituted with one or two of the same or different groups selected from: alkyl, haloalkyl, alkoxy, haloalkoxy, alkenyl, alkynyl and alkylamino radicals.

11. The compounds, compositions and uses according to claim 8 wherein the R¹ radical has an alpha-carbon (2-position in X) which is di-substituted with the same or different groups selected from: alkyl, haloalkyl, alkoxy, haloalkoxy, alkenyl, alkynl and alkylamino radicals.

12. The compounds, compositions and uses according to claim 10 wherein the alpha-carbon is chiral.

13. The compounds, compositions and uses according to claim 12 wherein the alpha-carbon has sp3 hybridised bonds.

14. The compounds, compositions and uses according to claim 12 wherein the alpha-carbon has essentially tetrahedral bond angles.

15. The pharmaceutical composition according to claim 3, wherein the compound is selected from the group consisting of:
(S)-3-hexadecanoylamino-caprolactam;
(S)-3-undecanoylamino-caprolactam;
(S)-3-(undec-10-enoyl)amino-caprolactam;
(S)-3-(undec-10-ynoyl)amino-caprolactam;
(S)-3-dodecanoylamino-caprolactam;
(S)-3-tetradecanoylamino-caprolactam;
(R)-3-hexadecanoylamino-caprolactam;
(S)-3-octadecanoylamino-caprolactam;
(S)-(Z)-3-(hexadec-9-enoyl)amino-caprolactam;
(S)-(Z)-3-(octadec-9-enoyl)amino-caprolactam;
(R)-(Z)-3-(octadec-9-enoyl)amino-caprolactam;
(S)-3-(2′,2′-dimethyl-dodecanoyl)amino-caprolactam;
(S)-3-(decyloxycarbonyl)amino-caprolactam;
(S)-(E)-3-(dodec-2-enoyl)amino-caprolactam;
(S)-3-(dec-9-enylaminocarbonyl)amino-caprolactam;
(S)-3-(decylaminocarbonyl)amino-caprolactam;
and pharmaceutically acceptable salts thereof.

16. The pharmaceutical composition according to claim 3, wherein the compound is selected from the group consisting of:
(R)-3-(2′,2′-Dimethyl-dodecanoyl)amino-caprolactam;
(S)-3-(2′,2′-Dimethyl-pentanoyl)amino-caprolactam;
(S)-3-(2′,2′-Dimethyl-pent-4-enoyl)amino-caprolactam;
(S)-3-(2′,2′-Dimethyl-propionyl)amino-caprolactam;
(S)-3-(2′,2′-Dimethyl-butyryl)amino-caprolactam;
(S,E)-3-(2′,2′-Dimethyl-dodec-4′-enoyl)amino-caprolactam;
(S)-3-(2′,2′,5′-Trimethyl-hex-4′-enoyl)amino-caprolactam;
(S)-3-(2′,2′,5′-Trimethyl-hexanoyl)amino-caprolactam;
(S)-3-(11′-bromo-undecanoyl)amino-caprolactam;
(S)-3-(11′-azido-undecanoyl)amino-caprolactam;
(S) Sodium 3-(undecanoyl)amino-caprolactam 11′-sulfonate tetrahydrate;
(S)-3-(Decanesulfonyl)amino-caprolactam;
(S)-3-(Dodecanesulfonyl)amino-caprolactam;
(S)-3-(Tetradecanesulfonyl)amino-caprolactam;
(S)-3-(Hexadecanesulfonyl)amino-caprolactam;
(S)-3-(Octadecanesulfonyl)amino-caprolactam;
and pharmaceutically acceptable salts thereof.

17. The pharmaceutical composition according to claim 3, wherein the compound is selected from the group consisting of: (S)-3-hexadecanoylamino-caprolactam, (S)-3-(2′,2′-dimethyl-dodecanoyl)amino-caprolactam, (S)-3-(2′,2′-dimethyl-propionyl)amino-caprolactam and pharmaceutically acceptable salts thereof.

18. The pharmaceutical composition according to claim 3, wherein the compound is selected from the group consisting of:
(S)-3-(2′-Propylpentanoyl)amino-caprolactam;
(3S,2′R) and (3S,2′S)-3-(2′-Ethylhexanoyl)amino-caprolactam;
(S)-3-(3′,3′-Dimethyldodecanoyl)amino-caprolactam;
(S)-(E)-3-(2′-Methyldodec-2′-enoyl)amino-caprolactam;
(3S,2′R) and (3S,2′S)-3-(2′-Methyldodecanoyl)amino-caprolactam;
(3S,2′S,3′R)-3-(3′-Hydroxy-2′-methyldecanoyl)amino-caprolactam;
(3S,2′R,3′S)-3-(3′-Hydroxy-2′-methyldecanoyl)amino-caprolactam;
(3S,3′R) and (3S,3′S)-3-(3′-Hydroxy-2′,2′-dimethyldecanoyl)amino-caprolactam;
(S)-(2′,2′-Dimethyl-3′-hydroxy-propionyl)amino-caprolactam;
(S)-(3′-Chloro-2′-(chloromethyl)-2′-methylpropionyl)amino-caprolactam;
and pharmaceutically acceptable salts thereof.

19. The use of a compound of formula (I) or (I′) according to claim 1 wherein the inflammatory disorder is selected from the group consisting of autoimmune diseases, vascular disorders, viral infection or replication, asthma, osteoporosis (low bone mineral density), tumor growth, rheumatoid arthritis, organ transplant rejection and/or delayed graft or organ function, a disorder characterised by an elevated TNF-α level, psoriasis, skin wounds, disorders caused by intracellular parasites, allergies, Alzheimer's disease, antigen induced recall response, immune response suppression, multiple sclerosis, ALS, fibrosis, and formation of adhesions.

20. The method of treatment, amelioration or prophylaxis of the symptoms of an inflammatory disease (including an adverse inflammatory reaction to any agent) by the administration to a patient of an anti-inflammatory amount of a compound, composition or medicament as claimed in claim 1.

21. The compounds, compositions, and uses of the compounds of general formula (I) or (I′), or their pharmaceutically acceptable salts, or a method of treatment according to claim 1, wherein the substituent R¹ is not a straight chain alkyl group.

22. The compounds, compositions, and uses of the compounds of general formula (I) or (I′), or their pharmaceutically acceptable salts, or a method of treatment according to claim 1, wherein the substituent R¹ is a branched chain alkyl group.

23. The compounds, compositions, and uses of the compounds of general formula (I) or (I′), or their pharmaceutically acceptable salts, or a method of treatment according to claim 1 wherein the substituent R¹ is not an alkyl group.

24. A pharmaceutical composition for treatment of an inflammatory disorder comprising, as active ingredient, (S,S) N,N′-bis-(2′-oxo-azepan-3′-yl) 2,2,6,6-tetramethylheptadiamide or a pharmaceutically acceptable salt thereof, and at least one pharmaceutically acceptable excipient and/or carrier.

25. A synthetic intermediate, useful in the synthesis of compounds of general formula (I) or (I′), selected from the group consisting of:
(E)-Methyl 2,2-dimethyl-dodec-4-enoate;
(E)-2,2-Dimethyl-dodec-4-enoyl chloride;
Methyl 2,2,5-trimethyl-hex-4-enoate;
2,2,5-Trimethyl-hex-4-enoyl chloride;
3,3-Dimethyldodecanoic acid;
3,3-Dimethyldodecanoyl chloride;
(E)-Ethyl 2-methyldodec-2-enoate;
(E)-2-Methyldodec-2-enoic acid;
(E)-2-Methyldodec-2-enoyl chloride;
(4S,2′S,3′R)-4-Benzyl-3-(3′-hydroxy-2′-methyldecanoyl)-oxazolidin-2-one;
(4R,2′R,3S)-4-Benzyl-3-(3′-hydroxy-2′-methyldecanoyl)-oxazolidin-2-one;
(2S,3R)-3-Hydroxy-2-methyldecanoic acid;
(2R,3S)-3-Hydroxy-2-methyldecanoic acid;
Methyl 2,2-dimethyl-3-hydroxy decanoate;
2,2-Dimethyl-3-hydroxy decanoic acid;
2,2-Dimethyl-3-(tetrahydropyran-2-yloxy)-propionic acid;
and pharmaceutically acceptable salts thereof.

26. The pharmaceutical composition according to claim 3, wherein the compound is (S)-3-(1′,1′-dimethylundecanesulfonyl)amino-caprolactam or a pharmaceutically acceptable salt thereof.
